SEQUENCE LISTING (50 sequences)

SEQ ID NO: 1 - DNA, 350 bp, Glycine max
  source 1..350: mol_type = genomic DNA
cttctccaacgactgtgcggccaactgaagctccatgttcaactgcagcccaaacgcctgcatcagaaactcacaagcatacctcaagggaaaaggaatacacctagcagaggtatggtggcaaataacaagcccccacaacctcatcgaagtgcggccaccaacaccttcctcatcattcccattgataataacagccatcaccaacgacgcagtcgagcccatgttagccatatactgagcatggcaaccgtgaggcgccctgagcgtggacccaaccaaacacagaggctgcacaagagcctcatcctgcaccaccctcacagcagaagcatgacaatccacaatca

SEQ ID NO: 2 - DNA, 350 bp, Glycine max
  source 1..350: mol_type = genomic DNA
cttctccaacgactgtgcggccaactgaagctccatgttcaactgcagcccaaacgcctgcatcagaaactcacaagcatacctcaagggaaaaggaatacacctagcagaggtatggtggcaaacaacaagcccccacaacctcatcgaagtgcggccaccaacaccttcctcatcattcccattgataataacagccatcaccaacgacgcagtcgagcccatgttagccatatactgagcatggcaaccgtgaggcgccctgagcgtggacccaaccaaacacagaggctgcacaagagcctcatcctgcaccaccctcacagcagaagcatgacaatccacaatca

SEQ ID NO: 3 - DNA, 16 bp, synthetic construct
  source 1..16: mol_type = other DNA
tggcaaataacaagcc

SEQ ID NO: 4 - DNA, 13 bp, synthetic construct
  source 1..13: mol_type = other DNA
gtggcaaacaaca

SEQ ID NO: 5 - DNA, 22 bp, synthetic construct
  source 1..22: mol_type = other DNA
gcctgcatcagaaactcacaag

SEQ ID NO: 6 - DNA, 21 bp, synthetic construct
  source 1..21: mol_type = other DNA
tgatgaggaaggtgttggtgg

SEQ ID NO: 7 - DNA, 20 bp, synthetic construct
  source 1..20: mol_type = other DNA
aagccagatcggtgagaaaa

SEQ ID NO: 8 - DNA, 20 bp, synthetic construct
  source 1..20: mol_type = other DNA
accagtgcatggtgatgatg

SEQ ID NO: 9 - DNA, 24 bp, synthetic construct
  source 1..24: mol_type = other DNA
tttttatttcttcttgctccaagt

SEQ ID NO: 10 - DNA, 27 bp, synthetic construct
  source 1..27: mol_type = other DNA
aaaatgcataacttcaacaatatctca

SEQ ID NO: 11 - DNA, 20 bp, synthetic construct
  source 1..20: mol_type = other DNA
gtgcggatttgatgatgttg

SEQ ID NO: 12 - DNA, 20 bp, synthetic construct
  source 1..20: mol_type = other DNA
agaaaatggggaagagggaa

SEQ ID NO: 13 - DNA, 21 bp, synthetic construct
  source 1..21: mol_type = other DNA
tccaatgctgaagctgaattt

SEQ ID NO: 14 - DNA, 21 bp, synthetic construct
  source 1..21: mol_type = other DNA
cgcgggatttttagaaagact

SEQ ID NO: 15 - DNA, 19 bp, synthetic construct
  source 1..19: mol_type = other DNA
agccaatcccaccagaaat

SEQ ID NO: 16 - DNA, 20 bp, synthetic construct
  source 1..20: mol_type = other DNA
tgctaacatgcacccagttt

SEQ ID NO: 17 - DNA, 20 bp, synthetic construct
  source 1..20: mol_type = other DNA
catcgaccttgccgtaaaat

SEQ ID NO: 18 - DNA, 20 bp, synthetic construct
  source 1..20: mol_type = other DNA
caaaggggttagtgctttgc

SEQ ID NO: 19 - protein, 110 aa, Glycine max
MEGGEEGLEMMVDSKDLQQQSKALDKLTDHVEDRQLDSTRVQEAMASIAASAEADRNAMRIREKELAAVKINAADVDIIANELELDKKVAERTLREHKGDAVAAIRHLLH

SEQ ID NO: 20 - protein, 1149 aa, Glycine max
MASASGAENSSVPPSPLPPPPPPQIHTSRTKLSHHHHNNNNNNNNNIDSTSKAIAQYTEDARLHAVFEQSGESGRSFDYSQSIRVTSESVPEQQITAYLLKIQRGGFIQPFGSMIAVDEPSFRILAYSDNARDMLGITPQSVPSLDDKNDAAFALGTDIRTLFTHSSAVLLEKAFSAREISLMNPIWIHSRTSGKPFYGILHRIDVGIVIDLEPARTEDPALSIAGAVQSQKLAVRAISQLQSLPGGDVKLLCDTVVESVRELTGYDRVMVYRFHEDEHGEVVAETKRPDLEPYIGLHYPATDIPQASRFLFKQNRVRMIVDCHASAVRVVQDEALVQPLCLVGSTLRAPHGCHAQYMANMGSTASLVMAVIINGNDEEGVGGRTSMRLWGLVVCHHTSARCIPFPLRYACEFLMQAFGLQLNMELQLAAQSLEKRVLRTQTLLCDMLLRDSPTGIVTQSPSIMDLVKCDGAALYYQGNYYPLGVTPTEAQIRDIIEWLLAFHRDSTGLSTDSLADAGYPGAASLGDAVCGMAVAYITEKDFLFWFRSHTAKEIKWGGAKHHPEDKDDGQRMHPRSSFKAFLEVVKSRSLPWENAEMDAIHSLQLILRDSFKDAEHSNSKAVLDPRMSELELQGVDELSSVAREMVRLIETATAPIFAVDVDGRINGWNAKVSELTGLPVEEAMGKSLVRDLVFKESEETVDKLLSRALKGEEDKNVEIKMRTFGPEHQNKAVFVVVNACSSKDYTNNVVGVCFVGQDVTGQKIVMDKFINIQGDYKAIVHNPNPLIPPIFASDDNTCCLEWNTAMEKLTGWSRADVIGKMLVGEVFGSCCQLKGSDSITKFMIVLHNALGGHDTDRFPFSFLDRYGKHVQAFLTANKRVNMDGQIIGAFCFLQIVSPELQQALKAQRQQEKNSFARMKELAYICQGVKNPLSGIRFTNSLLEATCLSNEQKQFLETSAACEKQMLKIIHDVDIESIEDGSLELEKGEFLLGNVINAVVSQVMLLLRERNLQLIRDIPEEIKTLAVYGDQLRIQQVLSDFLLNIVRYAPSPDGWVEIHVHPRIKQISDGLTLLHAEFRMVCPGEGLPPELIQNMFNNSGWGTQEGLGLSMSRKILKLMNGEVQYIREAQRCYFYVLLELPVTRRSSKKC

SEQ ID NO: 21 - protein, 1149 aa, Glycine max
MASASGAENSSVPPSPLPPPPPPQIHTSRTKLSHHHHNNNNNNNNNIDSTSKAIAQYTEDARLHAVFEQSGESGRSFDYSQSIRVTSESVPEQQITAYLLKIQRGGFIQPFGSMIAVDEPSFRILAYSDNARDMLGITPQSVPSLDDKNDAAFALGTDIRTLFTHSSAVLLEKAFSAREISLMNPIWIHSRTSGKPFYGILHRIDVGIVIDLEPARTEDPALSIAGAVQSQKLAVRAISQLQSLPGGDVKLLCDTVVESVRELTGYDRVMVYRFHEDEHGEVVAETKRPDLEPYIGLHYPATDIPQASRFLFKQNRVRMIVDCHASAVRVVQDEALVQPLCLVGSTLRAPHGCHAQYMANMGSTASLVMAVIINGNDEEGVGGRTSMRLWGLVICHHTSARCIPFPLRYACEFLMQAFGLQLNMELQLAAQSLEKRVLRTQTLLCDMLLRDSPTGIVTQSPSIMDLVKCDGAALYYQGNYYPLGVTPTEAQIRDIIEWLLAFHRDSTGLSTDSLADAGYPGAASLGDAVCGMAVAYITEKDFLFWFRSHTAKEIKWGGAKHHPEDKDDGQRMHPRSSFKAFLEVVKSRSLPWENAEMDAIHSLQLILRDSFKDAEHSNSKAVLDPRMSELELQGVDELSSVAREMVRLIETATAPIFAVDVDGRINGWNAKVSELTGLPVEEAMGKSLVRDLVFKESEETVDKLLSRALKGEEDKNVEIKMRTFGPEHQNKAVFVVVNACSSKDYTNNVVGVCFVGQDVTGQKIVMDKFINIQGDYKAIVHNPNPLIPPIFASDDNTCCLEWNTAMEKLTGWSRADVIGKMLVGEVFGSCCQLKGSDSITKFMIVLHNALGGHDTDRFPFSFLDRYGKHVQAFLTANKRVNMDGQIIGAFCFLQIVSPELQQALKAQRQQEKNSFARMKELAYICQGVKNPLSGIRFTNSLLEATCLSNEQKQFLETSAACEKQMLKIIHDVDIESIEDGSLELEKGEFLLGNVINAVVSQVMLLLRERNLQLIRDIPEEIKTLAVYGDQLRIQQVLSDFLLNIVRYAPSPDGWVEIHVHPRIKQISDGLTLLHAEFRMVCPGEGLPPELIQNMFNNSGWGTQEGLGLSMSRKILKLMNGEVQYIREAQRCYFYVLLELPVTRRSSKKC

SEQ ID NO: 22 - protein, 317 aa, Glycine max
MEVNEPEIDTITRNTSSEKESGQRKRGREGDKRIGVAAKLSSQLDRIIQTFESSVSAQDPTSITPCVEKLKDLLGLERGSELFYKAIRLMKKRANKITFVALEEPKLQLGWINMTDHRWVSEILNGHPIRCYQMFRMKKLVFLELCDILETKYNLKKTRNVSIYEQVGLFLYMLSQPGSVRNCKKRFQHSGETISRYFHSVLKVVCMFAKDIIKHVDPSFRDTLDEILKDARYHPYFRDCIGAIDGTHIRVCVPFHLQGVYIGRKNYTTTNVMAVCDFSMCFTFVWAGWEGSTHDTKIFMEALRKPALHFSYPPQGT

SEQ ID NO: 23 - protein, 594 aa, Glycine max
MATGQLFSRTTQSLFYNYKQLYIQRMLDFDFLCGRETPSVSGIISPGSEGFQKLFFGQEEIAIPVHATIEAACAAHPTADVFINFASFRSAAAASMAALKQPTIRVVAIIAEGVPESDTKQLVAYARANNKVVIGPATDGGIQAGAFKIGDTAGTIDNIIHCKLYRPGSVGFVSKSGGMSNELYNTIARVTDGIYEGIDIGGDAFPGSTLSDHVLRFNNIPQIKMMVVLGELGGRDEYSLVDALKQGKVSKPVVAWVDHEGAKSGSQLESAQAKNQALRDAGAVVPTSYETFEAAIKEAFDKLVEEGNITPVKETTPPPIPEDLNSAIKSGKVRAPTHIISTISDERGEEQCYAGVSMSSIIEEGYGVGGVVSLLWFKRSLPHYCTQFIEMCIMLCADHGPCASGAHNAIVTARAGKDLISSIVSGMLTIGPRFGGAIDDAARYFKDAHDRGLTPYEFVEGMKKKGIRVPGIGHRVKNKDNKDKRVELLQKFARTHFPCVKYMEYAIQVEDYTLSKANNLILNVDGAIASLFLDLFVGSGLFSKQEIDEIVEIGYLNGFFVLARCIGLIGHTFDQKRLKQPLYRHPWEDVLYTK

SEQ ID NO: 24 - protein, 608 aa, Glycine max
MATGQLFSRTTQALFYNYKQLPIQRMLDFDFLCGRETPSVAGIINPGSEGFQKLFFGQEEIAIPVHATIEAACAAHPTADVFINFASFRSAAASSMAALKQPTIRVVAIIAEGVPESDTKQLIAYARSNNKVVIGPATVGGIQAGAFKIGDTAGTIDNIIQCKLYRPGSVGFVSKSGGMSNELYNTIARVTDGIYEGIAIGGDVFPGSTLSDHVLRFNNMPQVKMMVVLGELGGRDEYSLVEALKQGKVSKPVVAWVSGTCARLFKSEVQFGHAGAKSGGELESAQAKNQALRDAGAVVPTSYEAFEAAIRETFDKLVEEGKITPIKEITPPPIPEDLSTAIKSGKVRAPTHIISTISDDRGEEPCYAGVPMSSIIEKGYGVGDVISLLWFKRSLPRYCTQFIEICIMLCADHGPCVSGAHNTIVTARAGKDLVSSLVSGLLTIGPRFGGAIDDAARYFKDAHDRALTPYEFVEGMKKKGIRVAGIGHRIKNRDNKDKRVELLQKFARTHFPSVKYMEYAVQVETYTLTKANNLVLNVDGAIGSLFLDLLAGSGMFTKQEIDEIVEIGYLNGLFVLARSIGLIGHTFDQKRLKQPLYRHPWEDVLYTK

SEQ ID NO: 25 - protein, 75 aa, Glycine max
MPKIRSYISYFTFFFLLFPSFSSPPSLPIYCHGRRTQQIYRHTYQQITTTSPLLLTYLLAGTHRRNALNLEYGER

SEQ ID NO: 26 - protein, 365 aa, Glycine max
MQSSTPAARNSKNKFITAKNVKSLVNTIVIPSISSTPLESLHRGNWPKLICGDAVDIRNLSLVYTLAGFDCIDCAADASFVSAVNEGIEAAKDIVCLRRPWVMISVNDDKDLHFRKAEFDPEDCPADCSRPCENVCPANAITFQGKSTSGISHNTEAPRVLKVQQCSIGLKMQDGVITESCYGCGRCLPVCPYNKIREVTYARDAITTAALIKRNCVDAMEIHTSGSDSSIDSKELWSALGDSVVYLKLIAVSASWHLSEEFILEIIRVSLPNGGFLHTIDGLKKEGLFQTTTEYLHDNMPTISSCDSSHALISGIAYGGYARKPTHNLYPIIYSIGIHAIEDHPEHLLMALKEALALLGPIKCL

SEQ ID NO: 27 - protein, 168 aa, Glycine max
MNMTITNSCPLIIFLCFMMPLFVFCDEGTHSGPSTQSLHSSHEDKNGSITVSMKVEHAHSASRKFWLHGTCTSKDISISQSQTSTPGIPQFIVQIVNNCVSGCAPSDIHLHCGMFASARMVNPRLFKRISYDDCLVNGGNPLAPSQIIRFTYSNTFSYPLAFKSAKFC

SEQ ID NO: 28 - protein, 97 aa, Glycine max
MSHTLSLSGDVRIVDLYTDTQNIAESGHPAVQLVLGFLWEMGLFREQIKGKAFRYHHFTAEGGNMQSKMELVYNYTRSDHSCPPATSRAPPATALSA

SEQ ID NO: 29 - protein, 1098 aa, Glycine max
MEGFDDDDFGELYAVDIEVPNAISEEEEEEKLDVNSNILNLDKDMDNNKVNEDCAAESDSDDDDDDLKIVLNDEDIPVGADACDDDGDGDNGDDNNGSRFFHPKSGRSRGLAILNNTKANASMGMGSYISSLNKGRRNGDTYIQNLALSSSRVCLAENPMAVQCGYGLALPWYWGIFDVNIDTLTEKPWKVPGVDITDYFNFGFNESSWKLYRASLEQLWRTSLQTGISVDDSTNWNQEAMREQTDQVVSGNAFFPSSDCGLPKGRAIQVEDSMVERKPTMDVRRPRNRDFNVIEIKLLDSSDDCSGSGNSTVMDASLEGESMAGSKRNALNSSAELNEVLSEDQLEDVKKAEDSSLQRRSGPIPGVDGDEHRDQADQHSEDTAEVPEGETEAEEGGGIDTCSSYPCWTESELSLGDQEHSLTSYTDGDSEATDNSVHVDNDKSLSPLKRKSLNCVTEMKESLALYWKNSKNNSINKKAVSAAYNSRTRGQFRKEWRNQSGGYEPHSYDMNKHTENDNDVSILKSSARNLSLLAHRPVDYGRHKDQLQVFGSHKRRDLSCNRETKQSYYYGGEKVIDELVSWRSKYYHEDRESFRENTNRYDRKNGDVGDYFFEPGPGFADSEDRDRDWYHLGCGHSSDDLCPCSYRESRQFPPKHSSFPDKERYTPRKRMDEKSLIERNCIDDFDECEFEFLNKSYRMSTVAEREQEFLDNNREEQFPHIYRDWRRSVRRGRRFDKPPLVLNNLCSGTMEVEDNCQKYTHFRTSNFKHRRQSYTDSVKNYAYGSRVNGNLGGSGRDKHARDNRDSNWSCDYTDTAEDEDFRICPVKEYQFYRSPSKFLNWTEDEIIFMRHETHATSLFTKVQSDDLPLQQHQLSMPKRDNEKYFKGSSKIMCRSKGGQAVLRCRKSVDLIHGEGKSQVRSSRVSCNGRLENVNQGIAKKRKRASVGFDESNKNTFKFDSPKYESNLKSKKWVQNLQDQAQKESSDIEEGQIVAEEPYMEKVSVSRRDASEGPAVTDSVNKKRMSQNENSSDQYIGGYDSQRILDSLAKMEKRRERFKQPMTMKKEAEESLKLNNDSIVDTGEMKQHRPTRKRRWVGN

SEQ ID NO: 30 - protein, 597 aa, Glycine max
MAFWWPLLVLALAFAICKFLLILIPPKVPSIDVDASDVLDDGSLTQENSFIYVPPRGTAQQSSGKVQCYEPATMKYLGYVPALTPDEVKEQVEKVRKAQKMWAKTSFKKRRHFLRILLKYIIKHQALICEISSRDTGKTMVDASLGEIMTTCEKINWLLSEGEQCLKPEYRSSGRAMLHKRSKVEFLPLGVIGAIVSWNYPFHNIFNPMLAAVFSGNGIVIKISEHASWSGCFYFRIIQSALAAIGAPEELVEVITGFAETGEALVASADKVIFVGSPGVGKMIMSNAAETLIPVTLELGGKDVFIVCEDADVDHVAQVAVRAALQSSGQNCAGAERFYVHRNIYASFVSKVTKIIKSVTAGPPLAGKYDMGALCMHAHSEKLEALINDALDKGAEIIARGSFGHIGEDAVDQYFPPTVIVNVNHSMRLMQEEAFGPIMPIMKFSSDEEVVRLANDSKYGLGCNVFSGSQSRAREIASQIHCGLAAVNDFAATYMCQSLPFGGVKNSGFGRFGGVEGLRACCLVKSVVEDRWWPFIKTVIPKPIQYPVAENGFEFQESLVEALYGLSVWDRLQALVNVLKMLTEQNSTSGSRKKKND

SEQ ID NO: 31 - DNA, 948 bp, Glycine max
  source 1..948: mol_type = genomic DNA
atgattcattcgaattgtgaaggttagggcatagaaagaaaggacggtggccggtggcggtgagtggtgtgtttgggaaagcgtaagagtaagaatggagggtggagaggaaggattggagatgatggtggactcaaaggacttacagcaacagagcaaagcgttggacaaactcactgatcacgtcgaggatcgccaactcgattccactcgcgtccaagaggccatggcttccatcgctgcttctgccgaagctgatcgcaatgccatgcgcatcagggaaaaagaattggctgctgttaagataaatgcagcagatgttgatataattgcaaatgaactagagttggacaaaaaggttgctgaaagaactttgcgtgagcataaaggtgatgcagttgctgctattcggcacttgcttcactagagaggtgggaatgaacaagagtgaatgctttagaaagttatatttatatgtatcagcgtatattgtttttcatcaaaattgaatctcaaatgtaactctgacctaattcattcagaaatcataactgtgttattgattgacagtttaccacccctccccctaaacttttctcaacttctgtgttaagaaaaggacccgttatctattggtattatctaatcatggagttaagtgcttcatgaggacctccacgtgtcaaaccatacatatgcttgctagttaagcatagatagagtttgccatcttcatgctcagcatgtgcttgtttcaaactcaaatagaaatccttgcttttgacatactcttgtagattgtgctctgtgatgcttgtgcaagtagcatctcctattttgtttttttcatccaaattgataaagctagatttcagatttgttgccagtgtatcttgaaatggacattgatggatagaagtgtccacactcttactaacttttgcttat

SEQ ID NO: 32 - DNA, 4607 bp, Glycine max
  source 1..4607: mol_type = genomic DNA
tcagaccacatagtcagaagaagccatcgagcaatcgcgtcgcgtgcgagagaaacgccaaacaacaaaagcagcagcagcgttactttgctgcacctgccattgtcactctgtctttcttctcttctctccttttttcacttttaccaatttagctcttccccctcccctctccaaaatttcacacaacaataatcataacttcatgaaatagttttaacaaaaaagagaaagagaagaagtgatagaaccaactttttctcttttctctgcagtggtggtgttaattaaattccaatggcttcagcaagcggagcggagaattcctccgtcccgccgtcgccgttgccgcctccgccgccgccgcaaatccacacctcgcggacgaagctgagccaccaccaccacaacaacaacaacaacaacaacaacaacatcgactccacgagcaaggccatcgcgcagtacacggaggacgcgcggctccacgccgtctttgagcagtccggcgagtccgggaggtcctttgactactcccaatcaatccgcgtcacatcggaatccgtcccggagcagcagataacggcttaccttctcaaaattcagcgcggcggcttcatccagcccttcggctccatgatcgccgtcgacgagccctccttccgcatccttgcctactccgacaacgcccgtgacatgctcggcattactccacagtccgtcccttcgctcgacgacaagaacgacgccgccttcgcgctcggaaccgatatccgaaccctcttcactcactccagcgccgttctcctcgaaaaggccttctccgcgcgcgaaattagcctcatgaaccctatctggattcactccagaacctccgggaagcctttctatggaatcctccaccgaattgacgtcggaattgtcatcgatttggagcctgcgcggacggaggatcctgccctctccatcgccggagctgtccagtcgcagaagctcgcggttcgcgcgatttcgcagcttcaatctctccccggtggcgatgttaagcttctttgtgatactgttgttgagagtgtcagggaattgacagggtatgatagggttatggtttataggtttcatgaggatgagcatggggaggttgttgctgagactaagaggcctgatttggagccttacattggattgcattatcccgctactgatattcctcaggcttctaggtttttgtttaagcagaatagggttaggatgattgtggattgtcatgcttctgctgtgagggtggtgcaggatgaggctcttgtgcagcctctgtgtttggttgggtccacgctcagggcgcctcacggttgccatgctcagtatatggctaacatgggctcgactgcgtcgttggtgatggctgttattatcaatgggaatgatgaggaaggtgttggtggccgcacttcgatgaggttgtgggggcttgttgtttgccaccatacctctgctaggtgtattccttttcccttgaggtatgcttgtgagtttctgatgcaggcgtttgggctgcagttgaacatggagcttcagttggccgcacagtcgttggagaagcgggttttgaggacacagactctgttgtgtgatatgcttctcagggactctcctactggcattgtaactcagagtcctagtattatggacttggtgaagtgtgacggagctgctctttattaccaagggaactattatccgttgggtgtgactccaactgaggctcagataagggatattattgagtggttgttggcctttcatagagattcgactggtttgagtactgatagtctggctgatgctggctatcctggggctgcctcgcttggggatgcagtttgtgggatggcggttgcttatattacagagaaggattttcttttctggttcaggtcgcacacggcgaaagagatcaaatggggtggtgcaaagcatcatcctgaggacaaggatgatgggcagagaatgcatccccgttcttccttcaaggcatttttagaagtggtgaaaagccgtagcttgccgtgggagaatgcggaaatggatgcaattcactctttgcagcttattctgcgtgactcgtttaaagatgctgagcatagcaattctaaggctgttttggatccccgtatgtcggaactagagttgcaaggggtcgatgaactaagttctgtagccagagagatggttagattgatcgaaacagccactgctccaatatttgctgttgatgttgatggccgcataaatgggtggaatgcaaaggtttcagaattgacaggactcccagttgaggaggctatggggaagtccttggttcgcgatcttgtgtttaaggagtctgaagaaactgtggacaagcttctttctcgtgctttaaaaggtgaagaagataagaatgttgagataaaaatgaggacgtttggcccagaacatcaaaataaggcagtttttgtagtggtgaatgcttgctccagcaaggattatacaaataatgtagttggagtgtgctttgttggtcaggatgttactggtcaaaaaattgtgatggacaaattcatcaacatacaaggcgactacaaggctattgtacataatccaaatcctttgatccctcccatttttgcatcggatgataacacgtgttgcttagagtggaacactgcaatggaaaagcttactggttggagccgcgcggatgtcattggaaaaatgttggtgggagaggttttcggcagttgctgtcagttgaagggttcagattcaataacaaagttcatgattgtcttacacaatgcgcttggtggacatgatacagatagattccctttttcatttcttgatcggtatggcaagcatgtgcaagctttcctgactgcaaataagagggttaacatggatggtcagatcattggggcattttgctttttgcaaattgtgagtccggaacttcaacaggctctgaaggcacagagacaacaagagaagaattcatttgctaggatgaaagagttagcttatatttgtcaaggagttaagaatcctttgagtggcatacgctttacaaactctcttttggaggctacatgcttgtccaatgagcaaaaacagtttcttgagactagtgctgcttgtgagaagcaaatgttaaagataatacacgatgttgatattgaaagcattgaggatgggtccctggagcttgaaaagggggaattcttgcttggaaatgtcataaatgcagttgttagccaagtaatgctactgttaagagaaagaaatttacagttgattcgtgatattcctgaagaaatcaagacattggctgtttatggtgatcaattgaggattcaacaagtgttgtctgatttcttattgaatatagtgcgctatgcaccatctccagatggctgggtagagattcatgtacatccaagaataaaacaaatctcagatgggctcactcttctccatgctgaatttagaatggtatgtcctggtgaaggtcttcctcctgaattgattcaaaacatgttcaataacagtgggtgggggactcaagaaggtttagggctgagcatgagcaggaagattctaaagctaatgaacggcgaagtgcagtatatcagggaggcccaacggtgctacttctatgttcttcttgaactacctgtgacacggagaagctctaaaaagtgttaaatatgtattctgtagctgtaagattaagtttaaaggtgctacttctatgttcttcttgaactacctgtgacacggagaagctctaaaaagtgttaaatatgtattctgtagctgtaagattaagtttaaagccctggcactgcccctcatgctgaccattagatagtttgttgtcatagattatgctctcacctaacaaggcaacttaagggacaagcgtttgaacaagctgatttggcaagctatcccagtcacatgtaattctaccaaattggactgttgttttgcaccttgacaaaacttgttcggtaaagcaagagtggccatttaatatgtagctaaatgtaaccttaaatcttactgataaagttatggctgagtagatgaaatgaagttttgccaatacgcacacaagtggagtctcctgtgaaaattttcaagcaaatgcttacatcattagcagctgcagtgaactccaatctatagttggattgaactgcatccatctcctgcactgttgtctgttctgatagtaactgcttataccaaatgcgttcccaagttggcaaataattgtaaaatcctagtctccaacaaggtttgccctctgcaatgttgctttccaactccacccttttttttttcatcattgcctgtaaatcccttcctactctgtaataaaagctgggttatttatcctgtaattttttaaatttcgtgaaagctagcttttcttcaataaaaaaatgaaatgcacttatctttctgtattatctttatttttgattctgtgtttctgagatttatcttattcctcaacacattagggagcgagtgaatggtttgatgagcttgaaagc

SEQ ID NO: 33 - DNA, 4607 bp, Glycine max
  source 1..4607: mol_type = genomic DNA
tcagaccacatagtcagaagaagccatcgagcaatcgcgtcgcgtgcgagagaaacgccaaacaacaaaagcagcagcagcgttactttgctgcacctgccattgtcactctgtctttcttctcttctctccttttttcacttttaccaatttagctcttccccctcccctctccaaaatttcacacaacaataatcataacttcatgaaatagttttaacaaaaaagagaaagagaagaagtgatagaaccaactttttctcttttctctgcagtggtggtgttaattaaattccaatggcttcagcaagcggagcggagaattcctccgtcccgccgtcgccgttgccgcctccgccgccgccgcaaatccacacctcgcggacgaagctgagccaccaccaccacaacaacaacaacaacaacaacaacaacatcgactccacgagcaaggccatcgcgcagtacacggaggacgcgcggctccacgccgtctttgagcagtccggcgagtccgggaggtcctttgactactcccaatcaatccgcgtcacatcggaatccgtcccggagcagcagataacggcttaccttctcaaaattcagcgcggcggcttcatccagcccttcggctccatgatcgccgtcgacgagccctccttccgcatccttgcctactccgacaacgcccgtgacatgctcggcattactccacagtccgtcccttcgctcgacgacaagaacgacgccgccttcgcgctcggaaccgatatccgaaccctcttcactcactccagcgccgttctcctcgaaaaggccttctccgcgcgcgaaattagcctcatgaaccctatctggattcactccagaacctccgggaagcctttctatggaatcctccaccgaattgacgtcggaattgtcatcgatttggagcctgcgcggacggaggatcctgccctctccatcgccggagctgtccagtcgcagaagctcgcggttcgcgcgatttcgcagcttcaatctctccccggtggcgatgttaagcttctttgtgatactgttgttgagagtgtcagggaattgacagggtatgatagggttatggtttataggtttcatgaggatgagcatggggaggttgttgctgagactaagaggcctgatttggagccttacattggattgcattatcccgctactgatattcctcaggcttctaggtttttgtttaagcagaatagggttaggatgattgtggattgtcatgcttctgctgtgagggtggtgcaggatgaggctcttgtgcagcctctgtgtttggttgggtccacgctcagggcgcctcacggttgccatgctcagtatatggctaacatgggctcgactgcgtcgttggtgatggctgttattatcaatgggaatgatgaggaaggtgttggtggccgcacttcgatgaggttgtgggggcttgttatttgccaccatacctctgctaggtgtattccttttcccttgaggtatgcttgtgagtttctgatgcaggcgtttgggctgcagttgaacatggagcttcagttggccgcacagtcgttggagaagcgggttttgaggacacagactctgttgtgtgatatgcttctcagggactctcctactggcattgtaactcagagtcctagtattatggacttggtgaagtgtgacggagctgctctttattaccaagggaactattatccgttgggtgtgactccaactgaggctcagataagggatattattgagtggttgttggcctttcatagagattcgactggtttgagtactgatagtctggctgatgctggctatcctggggctgcctcgcttggggatgcagtttgtgggatggcggttgcttatattacagagaaggattttcttttctggttcaggtcgcacacggcgaaagagatcaaatggggtggtgcaaagcatcatcctgaggacaaggatgatgggcagagaatgcatccccgttcttccttcaaggcatttttagaagtggtgaaaagccgtagcttgccgtgggagaatgcggaaatggatgcaattcactctttgcagcttattctgcgtgactcgtttaaagatgctgagcatagcaattctaaggctgttttggatccccgtatgtcggaactagagttgcaaggggtcgatgaactaagttctgtagccagagagatggttagattgatcgaaacagccactgctccaatatttgctgttgatgttgatggccgcataaatgggtggaatgcaaaggtttcagaattgacaggactcccagttgaggaggctatggggaagtccttggttcgcgatcttgtgtttaaggagtctgaagaaactgtggacaagcttctttctcgtgctttaaaaggtgaagaagataagaatgttgagataaaaatgaggacgtttggcccagaacatcaaaataaggcagtttttgtagtggtgaatgcttgctccagcaaggattatacaaataatgtagttggagtgtgctttgttggtcaggatgttactggtcaaaaaattgtgatggacaaattcatcaacatacaaggcgactacaaggctattgtacataatccaaatcctttgatccctcccatttttgcatcggatgataacacgtgttgcttagagtggaacactgcaatggaaaagcttactggttggagccgcgcggatgtcattggaaaaatgttggtgggagaggttttcggcagttgctgtcagttgaagggttcagattcaataacaaagttcatgattgtcttacacaatgcgcttggtggacatgatacagatagattccctttttcatttcttgatcggtatggcaagcatgtgcaagctttcctgactgcaaataagagggttaacatggatggtcagatcattggggcattttgctttttgcaaattgtgagtccggaacttcaacaggctctgaaggcacagagacaacaagagaagaattcatttgctaggatgaaagagttagcttatatttgtcaaggagttaagaatcctttgagtggcatacgctttacaaactctcttttggaggctacatgcttgtccaatgagcaaaaacagtttcttgagactagtgctgcttgtgagaagcaaatgttaaagataatacacgatgttgatattgaaagcattgaggatgggtccctggagcttgaaaagggggaattcttgcttggaaatgtcataaatgcagttgttagccaagtaatgctactgttaagagaaagaaatttacagttgattcgtgatattcctgaagaaatcaagacattggctgtttatggtgatcaattgaggattcaacaagtgttgtctgatttcttattgaatatagtgcgctatgcaccatctccagatggctgggtagagattcatgtacatccaagaataaaacaaatctcagatgggctcactcttctccatgctgaatttagaatggtatgtcctggtgaaggtcttcctcctgaattgattcaaaacatgttcaataacagtgggtgggggactcaagaaggtttagggctgagcatgagcaggaagattctaaagctaatgaacggcgaagtgcagtatatcagggaggcccaacggtgctacttctatgttcttcttgaactacctgtgacacggagaagctctaaaaagtgttaaatatgtattctgtagctgtaagattaagtttaaaggtgctacttctatgttcttcttgaactacctgtgacacggagaagctctaaaaagtgttaaatatgtattctgtagctgtaagattaagtttaaagccctggcactgcccctcatgctgaccattagatagtttgttgtcatagattatgctctcacctaacaaggcaacttaagggacaagcgtttgaacaagctgatttggcaagctatcccagtcacatgtaattctaccaaattggactgttgttttgcaccttgacaaaacttgttcggtaaagcaagagtggccatttaatatgtagctaaatgtaaccttaaatcttactgataaagttatggctgagtagatgaaatgaagttttgccaatacgcacacaagtggagtctcctgtgaaaattttcaagcaaatgcttacatcattagcagctgcagtgaactccaatctatagttggattgaactgcatccatctcctgcactgttgtctgttctgatagtaactgcttataccaaatgcgttcccaagttggcaaataattgtaaaatcctagtctccaacaaggtttgccctctgcaatgttgctttccaactccacccttttttttttcatcattgcctgtaaatcccttcctactctgtaataaaagctgggttatttatcctgtaattttttaaatttcgtgaaagctagcttttcttcaataaaaaaatgaaatgcacttatctttctgtattatctttatttttgattctgtgtttctgagatttatcttattcctcaacacattagggagcgagtgaatggtttgatgagcttgaaagc

SEQ ID NO: 34 - DNA, 1181 bp, Glycine max
  source 1..1181: mol_type = genomic DNA
ttcagaaataggtgggattctttgaaaaaggaatgacaattatgggctaagcttattgggaaggacataggtcttggttgggatgaggagaagaaaaccattgcagctagtgatgaatggtgggaagccaaaattcaacatatgcaccatttgaggattcaaggcaatatgaaggattcaacacaagagcataagagacaaatgataacattgatgataacactgacatggaagtgaatgagcctgagatagacacgataactcggaacacgtcttcggaaaaggaaagtggacagaggaagagaggtagagagggtgacaaaagaattggggttgctgccaagctttcctcacaattagatcgtattattcaaacatttgaatctagtgtatctgctcaagacccaactagcattactccatgtgtagaaaagttgaaagatctactaggacttgagcgtgggagtgaactattttacaaagccattagacttatgaaaaaaagggcaaacaagatcacctttgttgctttagaagagcctaagctacaacttggatggatcaacatgacagaccatcgttgggtatctgaaattctaaatggccatccaatacgttgttatcagatgtttagaatgaagaaacttgtctttcttgaattatgtgatatcttggaaaccaagtacaacttaaagaaaactcgaaatgtcagcatttatgagcaagttggcttgtttttatacatgttgagtcaaccaggttctgttcgtaattgtaagaaaagatttcaacattcaggtgaaacaatatctaggtatttccatagtgtcttaaaagttgtgtgtatgtttgcaaaagatataattaagcatgttgatccatcatttagagatactcttgatgagattctaaaagatgccagatatcacccttattttagggattgtattggtgcaatagatggtactcatatacgagtttgtgttccctttcatctacaaggagtctatattggtaggaaaaactacactaccactaatgtcatggctgtttgtgactttagcatgtgtttcacttttgtttgggcaggttgggaaggttctacccatgatactaagatatttatggaggctttacgtaagcctgcattgcatttttcatatcctcctcaaggtacttaa

SEQ ID NO: 35 - DNA, 2366 bp, Glycine max
  source 1..2366: mol_type = genomic DNA
ttttcttttttatccaatgcaatcaaaaggttcaccaaaaaagaataataggggtcacgtagcattacggctctctattattatttaaccgtgtgaactaacgaatcatattaccacaaattcagaacatactcttcagtcttcactttggtacttcacaagtatagctgggctttttttgtttgcttttccattacattttcaaactcttcatacaaattcatccaagcgtcactggtaatattccacttgtttgtaggaacggatcttgaaaatatttgagggatattaaagatctatctttttctttgtcatggccactggacaactattctcccgtactacacaatctttattttacaactacaagcaactttacatccagaggatgcttgattttgacttcctttgtggcagggaaacaccatcagtttctggaataattagccctggttctgagggatttcaaaaacttttttttggtcaggaggaaattgccatcccagttcatgctaccattgaagcagcttgtgctgcacatcctactgctgatgtcttcatcaactttgcttcatttagaagcgctgcagcagcatccatggctgccttgaagcagccaacgatcagagttgttgctattattgctgaaggagtacctgagtcagacactaagcaattggttgcgtatgcccgggcaaacaataaggttgttattggtccagccactgatggaggcattcaagcaggtgcttttaaaattggcgacacagctggaacaattgacaatataattcactgcaagctctataggccaggatctgttggatttgtatctaaatctggggggatgtccaatgaattatataacactatcgcccgcgtaactgatgggatttatgaaggcattgacattggaggagatgctttcccaggctccacactttctgaccatgttttacggtttaacaacataccacagattaaaatgatggtagtacttggagaacttggtgggcgtgatgagtattccttagtggacgccctaaaacaaggaaaagtatcaaaaccggttgtggcctgggttgatcatgaaggagctaaaagtggcagtcagttggagtctgctcaagcaaagaatcaggcactaagggacgctggagctgttgttcccacatcatatgaaacttttgaagctgcaattaaggaagcatttgacaagttggttgaagaagggaatatcacaccagtcaaggaaaccactcctccaccaatccccgaggaccttaactctgcaatcaagagtggaaaagtccgtgctccaactcatattatttccaccatttctgatgagagaggtgaggagcaatgctatgctggtgtatccatgtcttccattattgaagaaggttatggtgttggtggtgtggtctctcttttatggtttaaacgcagccttccccattactgtactcaatttattgagatgtgcatcatgctatgtgccgaccatggtccttgtgcctcaggtgctcacaatgctatcgtgacagcaagggccgggaaggacctaatttccagtattgtatcaggtatgcttacaattggtcctcgatttgggggtgccattgatgatgctgctcgctacttcaaggatgctcatgacagggggcttacgccttatgagtttgttgaaggtatgaaaaagaagggaattcgtgtgccaggaataggacacagggtaaagaataaggacaacaaagataagagagtagagctactacaaaagtttgcacgcacacatttcccttgcgtgaagtacatggaatatgctattcaagttgaagactacaccctctcaaaggcaaataatttaattctaaacgtggatggtgcaattgcatctcttttcttggatctttttgttggcagtggactgttcagcaaacaagagattgatgaaattgtggagattggctatctgaatggcttctttgtgctggcacgctgcattggtctgattgggcacacttttgaccaaaagcgattgaagcaaccactttaccgtcacccatgggaagatgttctctacacaaagtgaggaagttttgtctgactataagaactttgccatgcttcaaattgggaaatcatgcatctgaaataagctggagggattttttagctgttagactagtgttgtacgatccattggtgaaaagtcgtcttccaatcataacatgtattaactttcataatataaaaatacacatgtttttattatttcttattctcattgtaagaggtgagtatattggatttgtatgggagattttctacaataattacctatctctacttctttctta

SEQ ID NO: 36 - DNA, 2455 bp, Glycine max
  source 1..2455: mol_type = genomic DNA
ctaactcgagtctcgttttgtgcattttttttctccttccttcgatctaactaacacacacgacagagaaatgagaattgaagtaaaaattgtacagcgaagaagctaactaccaccctccacgtgactcgcgcaattcaactcacgcacactctcaacatcactatataaaccccctctcctccttcccttcatctttcagtctctcaccttccaactcgccctcgactcacttccttgtccgattccgccaacgaggccagtgaatcagatctatctgcttcttcgtcatggccaccggacaactattctcccgcactacacaggctttgttttacaactataagcaacttcccatacagcggatgcttgattttgacttcctttgcggcagggaaacaccatcagttgctggaataattaaccctggttctgagggatttcaaaaacttttttttggtcaggaggaaattgccatcccagttcatgccaccattgaagcagcttgtgctgcacatcctactgctgatgtcttcatcaactttgcttcatttagaagtgctgcagcatcatccatggcagctttgaagcagccaacaattagagttgttgctattattgctgaaggagtacccgagtcagacactaagcaattgattgcatatgcccgatcaaacaataaggttgttattggtccagccactgttggaggcattcaagcaggtgcttttaaaattggtgacacagctggaacaattgacaatataattcagtgcaagctctacaggcctggatctgttggatttgtttctaaatctggtgggatgtccaatgaactctacaacactattgcccgtgtaactgatgggatttatgaaggcattgctattggaggagatgttttccctggttccacactttctgaccatgttttacggtttaacaacatgccacaggttaaaatgatggtagtacttggggaacttggtgggcgtgatgagtattccttagtggaggccttaaaacaaggaaaggtatccaaaccggttgttgcctgggtcagtggcacatgtgcacgactcttcaaatctgaagtacaatttggtcatgcaggagccaaaagtggtggtgagttagagtctgctcaagcaaagaatcaggcactaagggacgctggagctgttgttcccacatcatatgaagcttttgaagctgcaattagggaaacatttgataaattggttgaagaagggaagatcacaccaatcaaggaaatcacacctccaccaatccctgaagaccttagtactgcaattaagagtggaaaagtccgtgctccaactcatattatttccaccatttctgatgacagaggagaggagccatgctatgctggtgtacccatgtcttccattatcgaaaaaggttatggtgttggtgatgttatctctcttttgtggtttaaacgaagccttccccgttactgtactcaatttattgagatatgcatcatgctatgtgccgaccatggtccttgtgtctctggtgctcacaatactattgtgacagcaagggcagggaaagacctagtttccagtctcgtatcaggtttgctaacaattggtcctcgatttgggggtgccatcgatgatgctgctcgctacttcaaggatgctcatgacagggcacttactccttatgagtttgttgaaggtatgaaaaagaagggaattcgtgtggcaggaatagggcacaggatcaagaatagggacaacaaagataagagagttgagctgctacagaagtttgcacgaacacattttccttccgtgaaatacatggaatatgctgttcaagttgagacctacacactcacaaaggcaaataatttagttcttaacgtagatggtgcaattggatctcttttcttggatcttcttgctggtagtggaatgttcaccaaacaagagattgatgaaattgtggagattggctatctgaatggcctctttgtgctggcacgctccattggtctgattggacacacctttgaccaaaagcgattgaagcaaccactttaccgtcacccatgggaggatgttctctacacaaagtgaggaagttttgtgcgactgtaagaactttcacatgcttcaaattagcaatcttttgggaaatcatgcatctgatataagctggagtgattttgtagctgttagactagtgttgtacgatctattggtgaaaaagtcatcttccatttgtaactttgattaaatttcataaagaaagaacttggtcattttttaaattacatttctcttgatgaatattctgatctttgtactgttttgacttcaataggaaattatcacataagttcgagcgcatttattctgcggtatggtatatgttcatggaaacaacaattttattgccagtgaatgaaatgctt

SEQ ID NO: 37 - DNA, 503 bp, Glycine max
  source 1..503: mol_type = genomic DNA
attttatgaataaagaaaacaaaataagatcgaagacaggacggaaaaacccactaaaaatctgaaaagaaatctcaccctgcatcccgtccgttgtcatgcctaaaataagatcatacatttcttattttacttttttttttcttttgtttccttctttttcatcaccaccgtcacttccaatctattgccatggtcgtcgcacccaacaaatctaccgccacacctaccaacaaatcacaaccacatcaccattacttcttacataccttcttgcagggacgcatcggagaaatgccttaaatttggaatatggcgaacgatagtggtcacagagcgttaaatatggaaagagtttgagagatcttagatctcagatctcattttgctcttttccgatatcggagtgatgaaaaaacgagaactagaacaaaaggttgtgggaggtggaaccatggtggtggtggttggaggagaggctttacaggtggggtaagagtttt

SEQ ID NO: 38 - DNA, 1098 bp, Glycine max
  source 1..1098: mol_type = genomic DNA
atgcagagttccacccctgctgcgagaaacagcaagaacaagtttattactgctaagaacgtgaagagcctcgtcaacaccatcgtgattccttccatttcatccactccactcgagtctctccacagaggcaactggcccaagctcatttgtggtgatgctgttgatatcagaaatctcagtctggtatacactcttgctggatttgactgcattgactgtgctgctgatgcatcgtttgttagtgctgtgaatgaaggaattgaagctgctaaagatattgtatgccttagaaggccctgggtgatgatcagtgtcaatgatgataaagatcttcattttcgtaaggccgaatttgatccagaggactgtccagcagactgttcacgaccttgtgaaaatgtgtgccctgcaaatgccataacattccaagggaaatctacttcaggaatttcacataatactgaagcacctagagtactgaaggtgcagcaatgttcaattggcttgaaaatgcaggacggagtcataacagaaagctgctatggttgtggtcgctgtcttccggtttgcccctacaataaaataagagaggtaacatatgcaagagatgctattacaactgctgctctcatcaagagaaattgtgttgatgctatggagatacatacaagtgggagtgattcttctatagacagcaaagaactctggagtgctctaggagattcagtagtatatttgaagcttatagctgtaagtgcctcttggcatttgagtgaagaatttattcttgaaataatccgggttagcttacctaatggtggttttcttcacacaattgatgggctgaaaaaagagggactcttccaaacaactactgaatacttacatgataatatgccaacaatcagctcatgtgattcatcacatgctttaattagcggtatagcttatggtggctacgcacggaagccgacacataatctataccccattatttacagtattgggatccatgcaattgaggatcatcccgagcatctcttgatggctcttaaggaagcacttgcattgttggggcctattaaatgtctatga

SEQ ID NO: 39 - DNA, 779 bp, Glycine max
  source 1..779: mol_type = genomic DNA
cattcctaaacacttgattttgattttgactatacttgtttgtgaagcagaaaaacaaagaaagatatcctagcagcataacactcagcaatgaacatgaccatcacaaactcttgtcctctgataatcttcctatgcttcatgatgcccctttttgtattttgtgacgagggaacgcattcaggaccatcgacccagagtttgcattcttctcatgaagacaaaaacgggtccataacagtgtcaatgaaagtagaacatgcacattctgcctctagaaaattttggttgcatggtacttgcactagcaaagacataagcatctcacaaagccaaacttctacaccaggaatcccacagttcatagtgcaaattgttaacaattgtgtttctggatgtgctcctagtgacatccacctccactgtggcatgtttgcttctgcaagaatggtgaacccaagattgttcaagaggatctcctatgatgattgcttagtgaatggagggaaccctttagcccctagccagatcatcagattcacatattccaataccttctcctaccctcttgcttttaagtctgccaaattttgctgagtattccaaaatatacaaatattcaaaagtagaataaaaaagctagcacagccttactagttactatactatatataccatgcctcagagaatccaatctctcatcaaaagggaatttctcctttccattggaaatgttggaattttcaagttaataaaatgtttccgtggttgtagctgca

SEQ ID NO: 40 - DNA, 1765 bp, Glycine max
  source 1..1765: mol_type = genomic DNA
ttcgaaggcaccatcttaaaaccgccttggaatgcttcaaaacaaagacggtcttgggaacaccgtcttaaatggttattatttttaattaatttatcgagcaaagtaagccactttctctctcattctcgcattttttcttgtcgactcaacttcttcatcgtggcgctgcgggagatgagggaggtggcgcctcaacttcttcatgtcacacactctctctcttagtggagacgtgaggattgttgacctttacactgatacgcagaacatagccgagagtgggcacccggcggtgcaattggtgctagggtttttgtgggaaatggggctttttagagagcaaatcaaggggaaggcctttcggtaccaccacttcaccgcagagggaggcaacatgcaatcgaagatggaattggtctacaactacacgaggagcgaccactcctgtcctccagccacgtcacgcgcccccccagctacggctttgtccgcatgacatcgttcacgcgaaaccgctcttgccccttgcacactaagaagaacctgtcgcgcagatataagaaataaaaaagaaaagaagaagaagaactttggtaccctcttctgcggcctctttctctctctctctcggttgtgagtgcagatttcttgcgtggtttatcatgggggatagtcagtattcattttctctcaccacatgtaggtattgttattgctcttaactttcaatttggtttaattttttgtttatattaaagagaagggtatgctttgtgtttcaactgattgcgctgtgttgtggaacaacccttctggaaagcttgttcagattgagcatgctttgacggttgttgggtctggacaaatgtctttgggaattaaaggtgattgattggttgaagtaactttaggtttcaaatttcacacctaagtttacagtagtgttctgggatttccgattaagttaagtgggattgaggtggctgagggtgctggtaaatagacagaaactggagtggtggtgtatgcggctatgatatattgagtttgacccttgtttatttaatagactggtggtgtgtggaaagatcactgaaaaaaattgtatgtgctggataagtattatggtggtgtgtggaacgatcactaaatacaatatcatcttagtgttagattatcataagaaagaaaaatgacagtttttttcccaccaaatgttgaaagaatgagcttgtatgggttgttttctaatagatcggtagaagattgaatcaatgcagaaaggattacaattctttgttataggccatttaaggggctttgacatgatgggaagttaagttctgttttgggaaaacagtgctctttgaaatgtagaatttcttcggttgagtatgctttagactttagaggaggctagagttagagatttcttagtttgtgatttaattgtgtattttggtgtaacttattttacatatttctgcatcggtccttaagtaagcactgatgtggaaaaacatttttttgacatcgttcacgtatgcaacaccgatgtagaaggtctaatttctacatcggtggtcaggatagcaccgttgtaaaaactgcaatgtaaaaaacgttgtctttctacaacgttcgtttgaaaaccgatgttgaaatttttaacttttaacgacatcctattcaacatcggttgaccaccgatgtagaatgtcgtatttcaccgatgtaaaaactgtgttatgtagtagtgaca

SEQ ID NO: 41 - DNA, 3720 bp, Glycine max
  source 1..3720: mol_type = genomic DNA
tagggtttaagattgtgcaagggttttggcaaaggaaaggttttttgttttgacaagtgagagatatttataagtcagttaggggttttgggtggcgtagccatttcatttcactactcttcttcctcaggtggctgcgatgtgaatataaactgaaaatggaaggtttcgacgatgacgatttcggcgaactctacgccgtcgacattgaagttcccaatgccatctcagaagaagaagaagaagaaaaactcgatgtaaacagtaacattttgaatcttgataaggatatggacaacaacaaagttaatgaggactgtgctgctgagagtgacagcgacgacgacgacgatgatctgaaaatcgtgctgaatgacgaggatattcctgtcggagctgatgcttgcgacgacgacggagatggcgacaatggtgatgataacaacggttccagattttttcatcctaagagtgggaggagtcgaggattggcgattttgaataatacgaaggccaatgcatccatgggtatgggttcttacatttcatccttgaataaagggaggaggaatggtgatacctacatccagaaccttgcattgagctcctctcgagtgtgtcttgcagaaaatcctatggctgttcaatgtggatatggattggctcttccttggtactggggtatatttgatgtaaacattgatacattaacggagaagccatggaaggttcctggagttgacataacagattactttaacttcggttttaatgagagctcttggaagctataccgtgcttcattggagcaactttggcgaacatccttgcaaactggtatttctgttgatgactctacaaactggaatcaggaagccatgagagagcaaactgatcaagttgtgtcaggaaatgcattttttccttcatcagattgtggattgccaaaaggcagagcaattcaggttgaggacagcatggttgaacggaaaccaaccatggatgtaaggcgtccacgtaatagggattttaatgttatagagatcaagctgcttgactcctctgatgactgttctggttctggcaatagcactgtaatggatgcatcattagaaggggaatctatggcaggtagtaagagaaatgctctcaattcttctgctgaactcaatgaagtgctgtctgaagatcagttggaagatgtgaaaaaagcagaggactcttctcttcagagaagaagtgggccaattcctggtgtagatggagatgagcatcgggatcaagcagaccaacattctgaagatactgcagaagtgccggagggagaaacagaagcagaggaaggtggtggcatagacacatgtagttcttatccatgttggactgaatcagaattgtctcttggagatcaagagcacagcctgacttcctatactgatggtgattctgaggcaacggataacagtgtacatgttgataatgataaaagtcttagccctttaaaaaggaaatcgttgaattgtgttactgaaatgaaggagtctttggcactttattggaagaactcaaagaacaacagtatcaataaaaaagcagtaagtgcagcttataattcaagaactagaggtcaattcagaaaggaatggaggaaccaaagtggtggatatgaacctcatagttatgatatgaataagcacactgaaaatgataatgatgtctccatcctcaagtcaagtgcaaggaatctgtctctgttggctcaccgacccgttgattatggcagacataaagaccaactgcaagtttttggcagtcataaaagaagagatctttcatgtaatagggaaacaaaacaatcctactattatggcggtgaaaaggtcattgatgagttggtttcatggcgctctaaatattatcacgaagatcgagaaagcttcagggagaatacaaatcgatatgacagaaaaaatggggatgtgggggattatttttttgaaccaggtcctggatttgcagatagtgaggacagagatagagattggtatcatcttggttgtgggcactcttctgatgacctgtgtccttgctcctacagggagtcaaggcagtttcctccaaagcattcatcttttcctgataaagagaggtatactccaaggaaaagaatggatgaaaaatcccttatagaaagaaattgcattgatgattttgatgaatgtgagtttgagtttctaaacaagagctatagaatgtccactgttgctgagagagaacaggaatttttagacaataatcgtgaagagcagtttccacatatttatagagattggagaagatctgtccgtaggggaagacgttttgacaagccccctttagttttgaataacttgtgttctgggacaatggaagtcgaagataattgtcagaaatatacacacttccggacttcaaattttaagcatcgtagacaatcttatacagattcagtgaaaaattatgcatatgggtcaagagtaaatggaaatcttgggggttcggggagagataagcatgctagggacaacagagacagcaactggtcgtgtgattatactgacactgctgaagatgaagatttccgaatttgtcctgtaaaagagtatcaattttataggtccccatctaagttcctgaactggacagaggatgaaattatttttatgcgtcatgaaactcatgcaacatctctgtttactaaggttcagagtgatgatttgccattgcagcagcatcagctaagtatgccaaaacgagataatgagaaatattttaaaggtagctctaagattatgtgcagaagtaagggtgggcaggcagtgctgagatgcaggaaatcagttgacttgattcatggggaaggaaagtctcaagtgagaagttccagagtctcgtgcaatggaaggcttgaaaatgtcaaccaggggattgctaagaaacggaagagagcttcagtgggttttgatgaatctaataaaaataccttcaaatttgactccccaaaatacgagagcaacctcaaaagtaagaaatgggtgcagaaccttcaagatcaagcgcagaaagaaagctcggacatcgaggaaggtcagattgttgcagaagaaccatacatggagaaggtttctgtatccagaagagatgcttctgaaggtccagcagtgactgacagtgtgaacaagaagagaatgtcacaaaatgaaaatagttctgaccaatatataggtggttacgacagtcaaaggattcttgattcattagccaagatggagaaacgtagggagcgctttaagcaaccaatgacaatgaaaaaagaagcagaagagagtttgaagcttaacaatgattccattgtagatacaggtgaaatgaagcaacacagaccaactcgaaagaggcgttgggttggtaattagcttttattgatggctaaagtttctttcagattccggacaagtaccatggtggaacttggttaatcatccttggtagaaactatattttggccttggcaaaattgatattatagatgttttcttttgacttggaataaagcctgattggcctaaagttttggtgtatttattgaaagctctatattgcgaagactgtaaataaccctgacatttatgagatatagtcaaatcgatttttagatgcctctagctcaggctccatcaa

SEQ ID NO: 42 - DNA, 2348 bp, Glycine max
  source 1..2348: mol_type = genomic DNA
atcaagacaatttcttctttcatcgcctaaagcttccccagattctttcactgtcttcaaccattgtcctcaaaacaacaatcgaaactccgcggaagcttctcgaaaccctaccgaggcgattttctcactcactccgaatctgctgccaagttccaatggcgttttggtggccattgctcgttctggcactcgctttcgccatctgcaagttccttctcattctcattcctcccaaggttccttccatcgacgtcgacgcttccgatgtgttggacgatgggagcctgacgcaggagaacagtttcatatacgtgcctccgaggggaacggcacagcaatcgagcggaaaagttcagtgctatgagcctgctaccatgaaatatttgggatatgttcctgcattgacgccggatgaggtcaaggagcaagtggaaaaagtacggaaggcacaaaaaatgtgggcgaagacaagcttcaagaaaaggcgccactttttacgtatacttttgaagtatataattaaacatcaagcacttatatgcgaaatatcttctcgcgatactggaaagacaatggtagatgcctctttaggagaaataatgacaacatgtgagaagataaattggctactatcagagggtgagcagtgcctaaagcctgagtaccggtcctctggaagagctatgcttcataagagatccaaagtagaatttctcccccttggtgttatcggtgccattgtgtcgtggaactatcctttccacaatatttttaaccctatgttggcagcagttttttctggaaatggcattgtgattaagatttcggaacatgcaagttggtctggatgcttttacttccgaatcatccaatcagcacttgctgccataggagctccggaggaacttgttgaggtgataacagggtttgctgaaacaggagaagcactggtagcttctgctgacaaagtcatttttgttggatcccctggggttggcaagatgataatgagcaatgctgctgagacacttatcccagttacactagagcttggtggaaaagatgtatttattgtttgtgaagatgcagatgtggatcatgttgctcaagttgctgtcagggctgctctgcagtcaagtgggcagaactgtgctggggcagagcgattttatgtccacaggaacatatatgcatcctttgtcagtaaagttaccaaaattataaaatctgttacagctggcccaccacttgctggaaagtatgatatgggagctttgtgtatgcatgcacattctgaaaagcttgaagcccttataaatgatgctttagacaaaggagctgaaattattgcacgtggaagttttggacatataggtgaagatgcagttgatcaatatttcccccctacagtgattgtgaatgtgaatcactcgatgagattaatgcaagaagaggcgtttggaccaatcatgccaataatgaaatttagctctgatgaagaggttgtcaggcttgcaaatgattcaaaatatgggcttgggtgtaatgttttctcaggcagtcagagccgtgctagagagatagcttcccagatacattgtgggcttgctgcggttaatgattttgctgcaacatacatgtgtcagtccctgccatttgggggtgtgaaaaacagtggatttggacgattcggtggtgttgagggtttgcgagcatgctgccttgtaaaatctgttgttgaagatagatggtggccattcataaaaactgtgataccgaagcctatacagtatcctgttgcagaaaatggatttgagttccaggagtcgcttgttgaagcactttacgggctcagcgtatgggaccgtttgcaagcattggtgaacgtgttgaaaatgcttactgagcagaactctacaagtggcagcagaaaaaagaaaaatgactaatcgtgtcaggacgggtaacattcctaatggttcatattcatatcatcatcatgttcaaataattgaggtcctttcggtttgtcgtttatctcccaagaagactccaactctaatttgtagtagccattattaactcctgccatttttgatgtgcttgttagatgacaatcctgggctacttgtctgatgaacgttcatatggattttatatcattatggtaatgaaattacagctagaaaataagtaactactatcatgccagaatcttttttttttaattagaaaactgaatataatctgtactgttaaattaccctcatcatcctcggagtagctcaaaaacactcccatgttatgaaatttgacattttactaatgattagattatcagattc

SEQ ID NO: 43 - DNA, 101 bp, Glycine max
  source 1..101: mol_type = genomic DNA
tttagaagagccaaatcatataattcatacatgctgcaaagatggataatkgtagttgtttacatatgtttaatatctaagctttccttcacttggtattg

SEQ ID NO: 44 - DNA, 100 bp, Glycine max
  source 1..100: mol_type = genomic DNA
tttgttccttccattattgaaaagtttaacctacgtgacaagtacatgtctttttttttttttttaattgtattatatatatacaataacttaacgtcgt

SEQ ID NO: 45 - DNA, 101 bp, Glycine max
  source 1..101: mol_type = genomic DNA
tttgttccttccattattgaaaagtttaacctacgtgacaagtacatgtcttttttttttttttttaattgtattatatatatacaataacttaacgtcgt

SEQ ID NO: 46 - DNA, 101 bp, Glycine max
  source 1..101: mol_type = genomic DNA
ggtagaattacatgtgactgggatagcttgccaaatcagcttgttcaaacrcttgtcccttaagttgccttgttaggtgagagcataatctatgacaacaa

SEQ ID NO: 47 - DNA, 101 bp, Glycine max
  source 1..101: mol_type = genomic DNA
cttcttagtgtgcaaggggcaagagcggtttcgcgtgaacgatgtcatgcrgacaaagccgtagctgggggggcgcgtgacgtggctggaggacaggagtg

SEQ ID NO: 48 - DNA, 101 bp, Glycine max
  source 1..101: mol_type = genomic DNA
agagagaacaggaatttttagacaataatcgtgaagagcagtttccacatrtttatagagattggagaagatctgtccgtaggggaagacgttttgacaag

SEQ ID NO: 49 - DNA, 101 bp, Glycine max
  source 1..101: mol_type = genomic DNA
tgtggaaaggatagttccacgacacaatggcaccgataacaccaagggggwgaaattctactttggatctcttatgaagcatagctcttccagaggacctg

SEQ ID NO: 50 - DNA, 101 bp, Glycine max
  source 1..101: mol_type = genomic DNA
ctagcctacataaactccgcacgacatagatttgcaattaatccgaattcraattttaaattccacatatataagcaaatcacaccgatcaagcaaaacac

(19) United States
(12) Patent Application Publication
(10) Pub. No.: US 2026/0223796 A1
(43) Pub. Date: Aug. 6, 2026

(54) COMPOSITIONS AND METHODS FOR MODIFYING SOYBEAN MATURITY

(71) Applicant: PIONEER HI-BRED INTERNATIONAL, INC., JOHNSTON, IA (US)

(72) Inventors: BEN BERGSMA, JOHNSTON, IA (US); LESLIE CHARLES KUHLMAN, LAWRENCE, KS (US); DONALD KYLE, PRINCETON, IL (US); JASON RAINES, CRAWFORDSVILLE, AR (US); JOHN BRYAN WOODWARD, ANKENY, IA (US); MING YANG, JOHNSTON, IA (US)

(73) Assignee: PIONEER HI-BRED INTERNATIONAL, INC., JOHNSTON, IA (US)

(21) Appl. No.: 19/153,015

(22) PCT Filed: Feb. 1, 2024
(86) PCT No.: PCT/US2024/014101

§ 371 Date: Aug. 1, 2025

Related U.S. Application Data
(60) Provisional application No. 63/482,888, filed on Feb. 2, 2023.

(51) Int. Cl. A01H 1/04; A01H 1/00; C12Q 1/6844; C12Q 1/6895

(52) U.S. Cl. CPC A01H 1/045; A01H 1/1215; C12Q 1/6844; C12Q 1/6895; C12Q 2600/13; C12Q 2600/172

(57) ABSTRACT

The present disclosure provides methods and compositions for producing, detecting and selecting soybean plants and seeds comprising a desired reproductive growth phenotype, such as early maturity, early flowering, late maturity or late flowering. Also provided are marker loci, marker alleles, primers, probes, and kits suitable for identifying and/or selecting soybean plants or soybean germplasms with one or more reproductive growth phenotypes.

### REFERENCE TO SEQUENCE LISTING SUBMITTED ELECTRONICALLY

The official copy of the sequence listing is submitted electronically via Patent Center as an XML formatted sequence listing with a file named 9390_SequenceListing created on Jan. 18, 2023 and having a size of 77,965 bytes and is filed concurrently with the specification. The sequence listing comprised in this XML formatted document is part of the specification and is herein incorporated by reference in its entirety.

### BACKGROUND

Soybean is a photoperiod sensitive plant that will rapidly flower and mature under short day-lengths. Through selective breeding for beneficial flowering, maturity phenotypes and naturally occurring native alleles that modify photoperiod sensitivity, soybean breeders have been able to create productive varieties within geographies beyond the original range of soybean cultivation. Soybean products are now classified and marketed within 13 maturity groups (MG), ranging from MG 000 in high northern latitudes to MG X in tropical latitudes. In order to maximize breeding efficiency, soybean breeders tend to cross parents with similar maturity behaviors to limit the number of progenies that would flower or mature outside of a desired maturity range. This practice limits the size of the effective breeding germplasm pool and over time decreases genetic diversity. Accordingly, the present disclosure provides compositions and methods to identify loci imparting maturity behavior variation allowing for the development of soybean that will thrive in a target environment.

### SUMMARY

Provided is a method for selecting soybean plants or soybean germplasm having an early maturity or early flowering phenotype comprising genotyping a soybean population comprising a plurality of soybean plants or soybean germplasm for the presence of at least one marker associated with early maturity or early flowering linked to or within a chromosomal interval flanked by and including marker loci BARCSOYSSR_15_0312 and BARCSOYSSR_15_1185, the chromosomal interval comprising a T at a locus corresponding to position 126 of SEQ ID NO: 1, and selecting from the soybean population one or more soybean plants or soybean germplasm comprising the at least one marker associated with early maturity or early flowering. The method can further include producing soybean plants or soybean germplasm having the early maturity or early flowering phenotype by crossing the selected soybean plant or soybean germplasm with a second soybean plant or soybean germplasm to produce a progeny population, wherein at least one soybean plant or soybean germplasm of the progeny population comprises the at least one marker associated with early maturity or early flowering and has an earlier maturity or flowering time (e.g., decreased number of days to maturity or flowering) as compared to a control plant.

Also provided is a method for producing soybean plants or soybean germplasm having an early maturity or early flowering phenotype comprising genotyping a population of soybean plants or soybean germplasm for the presence of one or more marker loci linked within 50 centimorgans (cM), 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of an early maturity or early flowering allele within a reproductive phenotype quantitative trait locus (QTL) flanked by and including BARCSOYSSR_15_0312 and BARCSOYSSR_15_1185 the early maturity or early flowering allele comprising allele T at marker locus S26383-001-Q001, selecting from the population one or more soybean plants or soybean germplasm comprising the one or more marker loci linked to the early maturity or early flowering allele, and crossing the selected soybean plant or soybean germplasm with a second soybean plant or soybean germplasm to produce a progeny population, wherein at least one soybean plant or soybean germplasm of the progeny population comprises the one or more marker loci associated with the early maturity or early flowering allele within the reproductive phenotype quantitative locus and has an earlier maturity or flowering time as compared to a control plant.

Further provided is a method for producing soybean plants or soybean germplasm having a late maturity or late flowering phenotype comprising genotyping a soybean population comprising a plurality of soybean plants or soybean germplasm for the presence of at least one marker associated with late maturity or late flowering linked to or within a chromosomal interval flanked by and including BARCSOYSSR_15_0312 and BARCSOYSSR_15_1185, the chromosomal interval comprising a C at a locus corresponding to position 126 of SEQ ID NO: 2, selecting from the soybean population one or more soybean plants or soybean germplasm comprising the at least one marker associated with late maturity or late flowering, and crossing the selected soybean plant or soybean germplasm with a second soybean plant or soybean germplasm to produce a progeny population, wherein at least one soybean plant or soybean germplasm of the progeny population comprises the at least one marker associated with late maturity or late flowering and has an later maturity or later flowering time (e.g., increased number of days to maturity or flowering) as compared to a control plant.

Also provided is a method for producing soybean plants or soybean germplasm having a late maturity or late flowering phenotype, the method comprising genotyping a population of soybean plants or soybean germplasm for the presence of one or more marker loci linked within 50 cM, 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of a late maturity or late flowering allele within a reproductive phenotype QTL flanked by and including BARCSOYSSR_15_0312 and BARCSOYSSR_15_1185 the late maturity or late flowering allele comprising allele C at marker locus S26383-001-Q001, selecting from the population one or more soybean plants or soybean germplasm comprising the one or more marker loci linked to the late maturity or late flowering allele, crossing the selected soybean plant or soybean germplasm with a second soybean plant or soybean germplasm to produce a progeny population, wherein at least one soybean plant or soybean germplasm of the progeny population comprises the one or more marker loci associated with the late maturity or late flowering allele within the reproductive phenotype quantitative locus and has an later maturity or later flowering time as compared to a control plant.

Provided is a method for producing a subpopulation of soybean plants or soybean germplasm having an early maturity or early flowering phenotype comprising crossing a first soybean plant or first soybean germplasm comprising an early maturity or early flowering allele T at marker locus S26383-001-Q001 within a chromosomal interval flanked by and including BARCSOYSSR_15_0312 and BARCSOYSSR_15_1185, with a second soybean plant or second soybean germplasm to form a soybean plant or soybean germplasm population, genotyping the soybean plant or soybean germplasm population for the presence of at least one marker associated with early maturity or early flowering linked to or within the chromosomal interval, and selecting from the soybean population one or more soybean plants or soybean germplasm comprising the at least one marker associated with early maturity or early flowering.

Also provided is a method for producing a subpopulation of soybean plants or soybean germplasm having a late maturity or late flowering phenotype comprising crossing a first soybean plant or first soybean germplasm comprising a late maturity or late flowering allele C at marker locus S26383-001-Q001 within a chromosomal interval flanked by and including BARCSOYSSR_15_0312 and BARCSOYSSR_15_1185, with a second soybean plant or second soybean germplasm to form a soybean plant or soybean germplasm population, genotyping the soybean plant or soybean germplasm population for the presence of at least one marker associated with late maturity or late flowering linked to or within the chromosomal interval, selecting from the soybean population one or more soybean plants or soybean germplasm comprising the at least one marker associated with late maturity or late flowering.

Further provided is a method for producing soybean plants or soybean germplasm having a desired maturity or flowering phenotype comprising genotyping a soybean population comprising a plurality of soybean plants or soybean germplasm for the presence of at least one marker associated with maturity or flowering linked to or within a chromosomal interval flanked by and including marker loci BARCSOYSSR_15_0312 and BARCSOYSSR_15_1185, wherein the chromosomal interval comprises a T or C at a marker locus S26383-001-Q001, the position corresponding to position 126 of SEQ ID NOs: 1 and 2, respectively, selecting from the soybean population one or more soybean plants or soybean germplasm comprising the at least one marker associated with maturity or flowering, and crossing the selected soybean plant or soybean germplasm with a second soybean plant or soybean germplasm to produce a progeny population, wherein at least one soybean plant or soybean germplasm of the progeny population comprises the at least one marker associated with maturity or flowering and wherein the at least one soybean plant of the progeny population has (i) an earlier maturity or earlier flowering time as compared to a control plant when the marker locus comprises a T at position 126 of SEQ ID NO: 1 and (ii) a later maturity or later flowering time as compared to a control plant when the marker locus comprises a C at position 126 of SEQ ID NO: 2.

### BRIEF DESCRIPTION OF THE SEQUENCE LISTING

The disclosure can be more fully understood from the following detailed description and the accompanying Sequence Listing, which form a part of this application. The sequence descriptions (Table 1) summarize the Sequence Listing attached hereto, which is hereby incorporated by reference and complies with the rules governing nucleotide and amino acid sequence disclosures in patent applications as set forth in 37 C.F.R. §§ 1.831-1.835.

TABLE 1
Sequence Listing Description

| SEQ | |
|---|---|
| ID NO: | Name |
| 1 | S26383-001-Q001 (Early) Early Maturity/ Early Flowering |
| | Allele |
| 2 | S26383-001-Q001 (Late) Late Maturity/ Late Flowering |
| | Allele |
| 3 | Early Maturity/ Early Flowering probe |
| 4 | Late Maturity/ Late Flowering Probe |
| 5 | S26383-001-Q001 Forward Primer |
| 6 | S26383-001-Q001 Reverse Primer |
| 7 | BARCSOYSSR_15_0312 Forward Primer |
| 8 | BARCSOYSSR_15_0312 Reverse Primer |
| 9 | BARCSOYSSR_15_0313 Forward Primer |
| 10 | BARCSOYSSR_15_0313 Reverse Primer |
| 11 | BARCSOYSSR_15_0731 Forward Primer |
| 12 | BARCSOYSSR_15_0731 Reverse Primer |
| 13 | BARCSOYSSR_15_0732 Forward Primer |
| 14 | BARCSOYSSR_15_0732 Reverse Primer |
| 15 | BARCSOYSSR_15_1184 Forward Primer |
| 16 | BARCSOYSSR_15_1184 Reverse Primer |
| 17 | BARCSOYSSR_15_1185 Forward Primer |
| 18 | BARCSOYSSR_15_1185 Reverse Primer |
| 19 | Glyma.15g139900 amino acid |
| 20 | Glyma.15g140000 amino acid - wild-type |
| 21 | Glyma.15g140000 amino acid - with SNP |
| 22 | Glyma.15g140100 amino acid |
| 23 | Glyma.15g140200 amino acid |
| 24 | Glyma.15g140300 amino acid |
| 25 | Glyma.15g140400 amino acid |
| 26 | Glyma.15g140500 amino acid |
| 27 | Glyma.15g140600 amino acid |
| 28 | Glyma.15g140700 amino acid |
| 29 | Glyma.15g140800 amino acid |
| 30 | Glyma.15g140900 amino acid |
| 31 | Glyma.15g139900 nucleic acid |
| 32 | Glyma.15g140000 nucleic acid - wild type |
| 33 | Glyma.15g140000 nucleic acid - with SNP |
| 34 | Glyma.15g140100 nucleic acid |
| 35 | Glyma.15g140200 nucleic acid |
| 36 | Glyma.15g140300 nucleic acid |
| 37 | Glyma.15g140400 nucleic acid |
| 38 | Glyma.15g140500 nucleic acid |
| 39 | Glyma.15g140600 nucleic acid |
| 40 | Glyma.15g140700 nucleic acid |
| 41 | Glyma.15g140800 nucleic acid |
| 42 | Glyma.15g140900 nucleic acid |
| 43-50 | See Table 5 |

### DETAILED DESCRIPTION

To limit the number of progenies that would flower or mature outside of a desired maturity range soybean breeders tend to cross parents with similar maturity behaviors. However, this practice can limit the size of the effective breeding germplasm pool which, over time, may decrease genetic diversity. Thus, methods for identifying and selecting alleles at loci imparting maturity behavior variation allows for the development of varieties that thrive in a target environment. Accordingly, the present disclosure provides methods and compositions for producing, detecting and selecting soybean plants and seeds having an early maturity and/or early flowering phenotype along with methods and compositions for producing, detecting and selecting soybean plants and seeds having a late maturity or late flowering phenotype.

Provided herein is a method for producing, detecting and/or selecting a soybean plant or soybean germplasm having a desired reproductive growth phenotype, such as early or late flowering or early or late maturity comprising genotyping a soybean population comprising a plurality of soybean plants or soybean germplasm for the presence of at least one marker associated with the desired reproductive phenotype linked to or within a chromosomal interval flanked by and including BARCSOYSSR_15_0312 and BARCSOYSSR_15_1185 and selecting from the soybean population one or more soybean plants or soybean germplasm comprising the at least one marker associated with the desired reproductive phenotype. and crossing the selected soybean plant or soybean germplasm with a second soybean plant or soybean germplasm to produce a progeny population in which at least one soybean plant or soybean germplasm of the progeny population comprises the at least one marker associated with the desired reproductive phenotype.

As used herein, “reproductive growth stage” “reproductive growth phenotype” or “reproductive stage” is a description of the characteristics associated with various phases of reproductive growth. “R” is the first reproductive growth stage when soybean begins to bloom by producing the first flower. Thus, as used herein flowering phenotype refers to the number of days to the initiation of flowering (R1), such that an early flowering phenotype refers to a decrease in the number of days (fewer days) between planting and the R1 stage, whereas a late flowering phenotype refers to an increase in the number of days (more days) between planting and the R1 stage. Similarly, when comparing two plants, the soybean plant or soybean variety that reaches the R1 stage first will be considered to have an earlier flowering time or earlier flowering phenotype, whereas the plant or variety that reaches the R1 stage second will be considered to have a later flowering time or later flowering phenotype. “R7” is the seventh reproductive growth stage when a soybean begins maturity. A soybean plant is identified as beginning maturity when it has one mature pod. “R8” is the eighth and final reproductive growth stage when a soybean is fully mature. A soybean plant is identified as fully mature when 95% of the pods are mature. Thus, as used herein maturity phenotype refers to the number of days to full maturity (R8), such that an early maturity phenotype refers to a decrease in the number of days (fewer days) between planting and the R8 stage, whereas a late maturity phenotype refers to an increase in the number of days (more days) between planting and the R8 stage. Similarly, when comparing two plants, the plant or variety that reaches the R8 stage first will be considered to have an earlier maturity or earlier maturity phenotype, whereas the plant or variety that reaches the R8 stage second will be considered to have a later maturity or later maturity phenotype.

In certain embodiments of the methods described herein, the plants and germplasm can have or be classified in an early maturity group of at least 000, 000, 00, 1, 2 or 3 and less than 6, 5, 4, 3, 2 or 1. In certain embodiments of the methods described herein, the plants and germplasm can have or be classified in an late maturity group of or at least 4, 5, 6, 7 or 8 and less than 10, 9, 8 or 7.

In certain embodiments, the desired reproductive growth phenotype is early maturity. In certain embodiments, the desired reproductive growth phenotype is early flowering. In certain embodiments, the desired reproductive growth phenotype is early maturity and early flowering.

In certain embodiments, the method for producing, detecting and/or selecting soybean plants or soybean germplasm having an early maturity or early flowering phenotype comprises genotyping a soybean population comprising a plurality of soybean plants or soybean germplasm for the presence of at least one marker linked to or associated with early maturity or early flowering linked to or within a chromosomal interval flanked by and including BARCSOYSSR_15_0312 and BARCSOYSSR_15_1185, a chromosomal interval flanked by and including BARCSOYSSR_15_0312 and BARCSOYSSR_15_1184, a chromosomal interval flanked by and including BARCSOYSSR_15_0312 and BARCSOYSSR_15_0732, a chromosomal interval flanked by and including BARCSOYSSR_15_0312 and BARCSOYSSR_15_0731, a chromosomal interval flanked by and including BARCSOYSSR_15_0313 and BARCSOYSSR_15_1185, a chromosomal interval flanked by and including BARCSOYSSR_15_0313 and BARCSOYSSR_15_1184, a chromosomal interval flanked by and including BARCSOYSSR_15_0313 and BARCSOYSSR_15_0732, or a chromosomal interval flanked by and including BARCSOYSSR_15_0313 and BARCSOYSSR_15_0731 and selecting from the soybean population one or more soybean plants or soybean germplasm comprising the at least one marker associated with early maturity or early flowering. In certain embodiments, the method further comprises crossing the selected soybean plant or soybean germplasm with a second soybean plant or soybean germplasm to produce a progeny population, wherein at least one soybean plant or soybean germplasm of the progeny population comprises the at least one marker associated with early maturity or early flowering linked to or within the chromosomal interval and has an earlier maturity or earlier flowering time as compared to a control plant (e.g., wild-type plant or a plant not comprising the at least one marker). In certain embodiments, the second soybean plant or soybean germplasm does not comprise the at least one marker associated with early maturity or early flowering. In certain embodiments, the second soybean plant or soybean germplasm is the same as the selected soybean plant or soybean germplasm, such that the crossing step comprises self-fertilization of the selected soybean plant or soybean germplasm. In certain embodiments, the chromosomal interval (e.g., the chromosomal interval flanked by and including BARCSOYSSR_15_0312 and BARCSOYSSR_15_1185) comprises a T at a locus corresponding to position 126 of SEQ ID NO: 1. In certain embodiments, the at least one marker linked to or associated with early maturity or early flowering is linked within 50 centimorgans (cM), 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of the chromosomal interval. In certain embodiments, the at least one marker linked to or associated with early maturity or early flowering comprises a marker within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of the chromosomal interval. In certain embodiments, at least 2 (e.g., 2, 3, 4, 5, 6, 7, 8, 9, 10, 20 or more) markers are detected. In certain embodiments, the at least 2 markers comprise a haplotype that is associated with early flowering or early maturity.

A cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency. Marker loci are themselves traits and can be assessed according to standard linkage analysis by tracking the marker loci during segregation. Thus, one cM is equal to a 1% chance that a marker locus will be separated from another locus, due to crossing over in a single generation.

In certain embodiments, the method for producing, detecting and/or selecting soybean plants or soybean germplasm having an early maturity or early flowering phenotype comprises genotyping a population of soybean plants or soybean germplasm for the presence of one or more marker loci linked within 50 cM, 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of an early maturity or early flowering allele within a reproductive growth phenotype quantitative trait locus (QTL) flanked by and including BARCSOYSSR_15_0312 and BARCSOYSSR_15_1185, a reproductive growth phenotype QTL flanked by and including BARCSOYSSR_15_0312 and BARCSOYSSR_15_1184, a reproductive growth phenotype QTL flanked by and including BARCSOYSSR_15_0312 and BARCSOYSSR_15_0732, a reproductive growth phenotype QTL flanked by and including BARCSOYSSR_15_0312 and BARCSOYSSR_15_0731, a reproductive growth phenotype QTL flanked by and including BARCSOYSSR_15_0313 and BARCSOYSSR_15_1185, a reproductive growth phenotype QTL flanked by and including BARCSOYSSR_15_0313 and BARCSOYSSR_15_1184, a reproductive growth phenotype QTL flanked by and including BARCSOYSSR_15_0313 and BARCSOYSSR_15_0732, or a reproductive growth phenotype QTL flanked by and including BARCSOYSSR_15_0313 and BARCSOYSSR_15_0731 and selecting from the population one or more soybean plants or soybean germplasm comprising the one or more marker loci linked to the early maturity or early flowering allele. In certain embodiments, the early maturity or early flowering allele comprises allele T at marker locus S26383-001-Q001. In certain embodiments the method further comprises, crossing the selected soybean plant or soybean germplasm with a second soybean plant or soybean germplasm to produce a progeny population, wherein at least one soybean plant or soybean germplasm of the progeny population comprises the one or more marker loci associated with the early maturity or early flowering allele within the reproductive phenotype QTL. In certain embodiments, the second soybean plant or soybean germplasm does not comprise the at least one marker associated with early maturity or early flowering. In certain embodiments, the second soybean plant or soybean germplasm is the same as the selected soybean plant or soybean germplasm, such that the crossing step comprises self-fertilization of the selected soybean plant or soybean germplasm. In certain embodiments, the one or more marker loci are within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of the early maturity or early flowering allele within the reproductive phenotype QTL. In certain embodiments, at least 2 (e.g., 2, 3, 4, 5, 6, 7, 8, 9, 10, 20 or more) markers are detected. In certain embodiments, the at least 2 markers comprise a haplotype that is associated with early flowering or early maturity.

In certain embodiments of the methods for producing, detecting and/or selecting soybean plants or soybean germplasm having an early maturity or early flowering phenotype described herein, the at least one marker or marker loci associated with early maturity or early flowering or linked to the early maturity or early flowering allele is selected from the group consisting of a T at position 11441207 on Chr15 (marker locus S26383-001-Q001), a G at position 11430145 on Chr15, a 1 bp deletion of a T at position 11434182 on Chr15, an A at position 11436193 on Chr15, an A at position 11522371 on Chr15, a G at position 11529959 on Chr15, a T at position 11542556 on Chr15, a G at position 11545315 on Chr15 or any combination thereof. In certain embodiments, the at least one marker comprises a T at marker locus S26383-001-Q001 (T at a locus corresponding to position 126 of SEQ ID NO: 1).

As used herein, “haplotype” refers to a combination of alleles present within a particular plant's genome at two or more linked marker loci, for instance at two or more loci on a particular linkage group.

In certain embodiments, the desired reproductive growth phenotype is late maturity. In certain embodiments, the desired reproductive growth phenotype is late flowering. In certain embodiments, the desired reproductive growth phenotype is late maturity and late flowering.

In certain embodiments, the method for producing, detecting and/or selecting soybean plants or soybean germplasm having a late maturity or late flowering phenotype comprises genotyping a soybean population comprising a plurality of soybean plants or soybean germplasm for the presence of at least one marker associated with late maturity or late flowering linked to or within a chromosomal interval flanked by and including BARCSOYSSR_15_0312 and BARCSOYSSR_15_1185, a chromosomal interval flanked by and including BARCSOYSSR_15_0312 and BARCSOYSSR_15_1184, a chromosomal interval flanked by and including BARCSOYSSR_15_0312 and BARCSOYSSR_15_0732, a chromosomal interval flanked by and including BARCSOYSSR_15_0312 and BARCSOYSSR_15_0731, a chromosomal interval flanked by and including BARCSOYSSR_15_0313 and BARCSOYSSR_15_1185, a chromosomal interval flanked by and including BARCSOYSSR_15_0313 and BARCSOYSSR_15_1184, a chromosomal interval flanked by and including BARCSOYSSR_15_0313 and BARCSOYSSR_15_0732, or a chromosomal interval flanked by and including BARCSOYSSR_15_0313 and BARCSOYSSR_15_0731 and selecting from the soybean population one or more soybean plants or soybean germplasm comprising the at least one marker linked to or associated with late maturity or late flowering. In certain embodiments, the method further comprises crossing the selected soybean plant or soybean germplasm with a second soybean plant or soybean germplasm to produce a progeny population, wherein at least one soybean plant or soybean germplasm of the progeny population comprises the at least one marker associated with late maturity or late flowering linked to or within the chromosomal interval and has a later maturity or flowering time as compared to a control plant. In certain embodiments, the second soybean plant or soybean germplasm does not comprise the at least one marker associated with late maturity or late flowering. In certain embodiments, the second soybean plant or soybean germplasm is the same as the selected soybean plant or soybean germplasm, such that the crossing step comprises self-fertilization of the selected soybean plant or soybean germplasm. In certain embodiments, the chromosomal interval (e.g., the chromosomal interval flanked by and including BARCSOYSSR_15_0312 and BARCSOYSSR_15_1185) comprises a C at a locus corresponding to position 126 of SEQ ID NO: 2. In certain embodiments, the at least one marker linked to or associated with late maturity or late flowering is linked within 50 cM, 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of the chromosomal interval. In certain embodiments, the at least one marker linked to or associated with late maturity or late flowering comprises a marker locus within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of the chromosomal interval. In certain embodiments, at least 2 (e.g., 2, 3, 4, 5, 6, 7, 8, 9, 10, 20 or more) markers are detected. In certain embodiments, the at least 2 markers comprise a haplotype that is associated with late flowering or late maturity.

In certain embodiments, the method for producing, detecting and/or selecting soybean plants or soybean germplasm having a late maturity or late flowering phenotype comprises genotyping a population of soybean plants or soybean germplasm for the presence of one or more marker loci linked within 50 cM, 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of a late maturity or late flowering allele within a reproductive phenotype QTL flanked by and including BARCSOYSSR_15_0312 and BARCSOYSSR_15_1185, a reproductive growth phenotype QTL flanked by and including BARCSOYSSR_15_0312 and BARCSOYSSR_15_1184, a reproductive growth phenotype QTL flanked by and including BARCSOYSSR_15_0312 and BARCSOYSSR_15_0732, a reproductive growth phenotype QTL flanked by and including BARCSOYSSR_15_0312 and BARCSOYSSR_15_0731, a reproductive growth phenotype QTL flanked by and including BARCSOYSSR_15_0313 and BARCSOYSSR_15_1185, a reproductive growth phenotype QTL flanked by and including BARCSOYSSR_15_0313 and BARCSOYSSR_15_1184, a reproductive growth phenotype QTL flanked by and including BARCSOYSSR_15_0313 and BARCSOYSSR_15_0732, or a reproductive growth phenotype QTL flanked by and including BARCSOYSSR_15_0313 and BARCSOYSSR_15_0731 and selecting from the population one or more soybean plants or soybean germplasm comprising the one or more marker loci linked to the late maturity or late flowering allele. In certain embodiments, the late maturity or late flowering allele comprises allele C at marker locus S26383-001-Q001. In certain embodiments the method further comprises, crossing the selected soybean plant or soybean germplasm with a second soybean plant or soybean germplasm to produce a progeny population, wherein at least one soybean plant or soybean germplasm of the progeny population comprises the one or more marker loci linked to the late maturity or late flowering allele within the reproductive phenotype QTL. In certain embodiments, the second soybean plant or soybean germplasm does not comprise the at least one marker associated with late maturity or late flowering. In certain embodiments, the second soybean plant or soybean germplasm is the same as the selected soybean plant or soybean germplasm, such that the crossing step comprises self-fertilization of the selected soybean plant or soybean germplasm. In certain embodiments, the one or more marker loci are within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of the late maturity or late flowering allele within the reproductive phenotype QTL. In certain embodiments, at least 2 (e.g., 2, 3, 4, 5, 6, 7, 8, 9, 10, 20 or more) markers are detected. In certain embodiments, the at least 2 markers comprise a haplotype that is associated with late flowering or late maturity.

In certain embodiments of the methods for producing, detecting and/or selecting soybean plants or soybean germplasm having a late maturity or late flowering phenotype described herein, the at least one marker associated with late maturity or late flowering or linked to the late maturity or late flowering allele is selected from the group consisting of a C at position 11441207 on Chr15 (marker locus S26383-001-Q001), a T at position 11430145 on Chr15, a T at position 11434182 on Chr15, a G at position 11436193 on Chr15, a G at position 11522371 on Chr15, an A at position 11529959 on Chr15, an A at position 11542556 on Chr15, an A at position 11545315 on Chr15 or any combination thereof. In certain embodiments, the at least one marker comprises a C at marker locus S26383-001-Q001 (C at a locus corresponding to position 126 of SEQ ID NO: 2).

As used herein, “chromosome interval”, “chromosomal interval” and the like refers to a chromosome segment defined by specific flanking marker loci. The term “chromosome segment” designates a contiguous linear span of genomic DNA that resides in planta on a single chromosome. In certain embodiments, the chromosomal intervals of the methods described herein comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL.

The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question.

As used herein, “marker” or “molecular marker” “marker loci” or “marker locus” denotes a nucleic acid sequence that is sufficiently unique to characterize a specific locus on the genome. Any detectable polymorphic trait can be used as a marker so long as it is inherited differentially and exhibits linkage disequilibrium with a phenotypic trait of interest. Examples of markers for use in the methods described herein, include, but are not limited to, simple sequence repeats (SSRs), single nucleotide polymorphisms (SNPs), restriction fragment length polymorphisms (RFLPs), and indels. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD). The method for genotyping for the presence (i.e., detecting) of the marker or marker loci described herein may be any method described herein or known in the art.

“Amplifying,” in the context of nucleic acid amplification, is any process whereby additional copies of a selected nucleic acid (or a transcribed form thereof) are produced. In some embodiments, an amplification-based marker technology is used wherein a primer or amplification primer pair is admixed with genomic nucleic acid isolated from the first plant or germplasm, and wherein the primer or primer pair is complementary or partially complementary to at least a portion of the marker locus and is capable of initiating DNA polymerization by a DNA polymerase using the plant genomic nucleic acid as a template. The primer or primer pair is extended in a DNA polymerization reaction having a DNA polymerase and a template genomic nucleic acid to generate at least one amplicon. In other embodiments, plant RNA is the template for the amplification reaction.

As used herein, a “single nucleotide polymorphism (SNP)” refers to a DNA sequence variation occurring when a single nucleotide—A, T, C or G—in the genome (or other shared sequence) differs between members of a biological species or paired chromosomes in an individual.

As used herein “allele” refers to any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant. A “favorable allele” as used herein refers to the allele at a particular locus (a marker, a QTL, a gene etc.) that confers, or contributes to, an agronomically desirable phenotype, e.g., early maturity or late maturity, and that allows the identification of plants with that agronomically desirable phenotype. A favorable allele of a marker is a marker allele that segregates with the favorable phenotype. An “unfavorable allele” of a marker is a marker allele that segregates with the unfavorable plant phenotype, therefore providing the benefit of identifying plants that can be removed from a breeding program or planting.

As used herein, the term “crossing”, “crossed”, “cross” or the like refers to a sexual cross and involved the fusion of two haploid gametes via pollination to produce diploid progeny (e.g., cells, seeds or plants). The term encompasses both the pollination of one plant by another and selfing (or self-pollination (also referred to herein as self-fertilization), e.g., when the pollen and ovule are from the same plant). The plants of the crosses in the methods described herein may be elite soybean plants, exotic soybean plants, or a combination thereof.

As used herein, and “elite line” is an agronomically superior line that has resulted from many cycles of breeding and selection for superior agronomic performance. Numerous elite lines are available and known to those of skill in the art of soybean breeding. As used herein, an “exotic soybean line” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like.

As used herein, the term “germplasm” refers to genetic material of or from an individual (e.g., a plant), a group of individuals (e.g., a plant line, variety or family), or a clone derived from a line, variety, species, or culture, or more generally, all individuals within a species or for several species (e.g., soybean germplasm collection). The germplasm can be part of an organism, cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, that can be cultured into a whole plant.

As used herein, the term “linked to” in connection with a relationship between a marker locus, QTL, or chromosomal interval and a phenotype refers to a statistically significant dependence of marker frequency with respect to a quantitative scale or qualitative gradation of the phenotype. Thus, an allele of a marker is linked with a trait of interest when the allele of the marker locus and the trait phenotypes are found together in the progeny of an organism more often than if the marker genotypes and trait phenotypes segregated separately. When a trait is stated to be linked to a given marker it will be understood that the actual DNA segment whose sequence affects the trait generally co-segregates with the marker.

In certain embodiments, the markers of the methods described herein are closely linked to the chromosomal intervals or alleles described herein. As used herein, “closely linked” means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., high seed protein content). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

A “genetic map” is the relationship of genetic linkage among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a physical map of the genome refers to absolute distances (for example, measured in base pairs or isolated and overlapping contiguous genetic fragments, e.g., contigs). A physical map of the genome does not take into account the genetic behavior (e.g., recombination frequencies) between different points on the physical map. A “genetic recombination frequency” is the frequency of a crossing over event (recombination) between two genetic loci. Recombination frequency can be observed by following the segregation of markers and/or traits following meiosis. In some cases, two different markers can have the same genetic map coordinates. In that case, the two markers are in such close proximity to each other that recombination occurs between them with such low frequency that it is undetected.

Genetic maps are graphical representations of genomes (or a portion of a genome such as a single chromosome) where the distances between markers are measured by the recombination frequencies between them. Plant breeders use genetic maps of molecular markers to increase breeding efficiency through marker-assisted selection, a process where selection for a trait of interest is not based on the trait itself but rather on the genotype of a marker linked to the trait. A molecular marker that demonstrates reliable linkage with a phenotypic trait provides a useful tool for indirectly selecting the trait in a plant population, especially when accurate phenotyping is difficult, slow, or expensive.

Also provided herein are methods for introgressing a desired reproductive growth phenotype and for producing a subpopulation of soybean plants or soybean germplasm having a desired reproductive growth phenotype, such as early or late flowering or early or late maturity comprising crossing a first soybean plant or first soybean germplasm comprising a desired reproductive growth phenotype allele within a chromosomal interval described herein (e.g., a chromosomal interval flanked by and including BARCSOYSSR_15_0312 and BARCSOYSSR_15_1185) with a second soybean plant or second soybean germplasm to form a soybean plant or soybean germplasm population, genotyping one or more plants of the soybean plant or soybean germplasm population for the presence of at least one marker associated with the desired reproductive phenotype linked to or within the chromosomal interval, and selecting from the soybean population one or more soybean plants or soybean germplasm comprising the at least one marker associated with the desired reproductive phenotype.

“Introgressing”, “introgression” and the like, as used herein, refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, such as those described herein, at a QTL, a transgene, or the like. Offspring comprising the desired allele may be repeatedly backcrossed to a line having a desired genetic background and selected for the desired allele, to result in the allele becoming fixed in a selected genetic background. The process of “introgressing” is often referred to as “backcrossing” when the process is repeated two or more times.

In certain embodiments, the desired reproductive growth phenotype is early maturity or early flowering. In certain embodiments, the methods for introgressing an early maturity or early flowering phenotype and for producing a subpopulation of soybean plants or soybean germplasm having an early maturity or early flowering comprises crossing a first soybean plant or first soybean germplasm comprising an early maturity or early flowering allele T at marker locus S26383-001-Q001 (position 126 of SEQ ID NO: 1) within a chromosomal interval described herein (e.g., a chromosomal interval flanked by and including BARCSOYSSR_15_0312 and BARCSOYSSR_15_1185), with a second soybean plant or second soybean germplasm to form a soybean plant or soybean germplasm population, genotyping the soybean plant or soybean germplasm population for the presence of at least one marker associated with early maturity or early flowering linked to or within the chromosomal interval, and selecting from the soybean population one or more soybean plants or soybean germplasm comprising the at least one marker associated with early maturity or early flowering. In certain embodiments, the second soybean plant does not comprise the early maturity or early flowering allele T at marker locus S26383-001-Q001. In certain embodiments, the second soybean plant or soybean germplasm comprises a late maturity or late flowering allele C at marker locus S26383-001-Q001 within the chromosomal interval. In certain embodiments, the second soybean plant or soybean germplasm is the same as the first soybean plant or soybean germplasm, such that the crossing step comprises self-fertilization of the first soybean plant or soybean germplasm. In certain embodiments, the one or more soybean plants selected has an earlier maturity and/or an earlier flowering time as compared to the second soybean plant or soybean germplasm.

In certain embodiments, the at least one marker associated with early maturity or early flowering linked to or within the chromosomal interval is linked within 50 centimorgans (cM), 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of the chromosomal interval. In certain embodiments, the at least one marker associated with early maturity or early flowering linked to or within the chromosomal interval is linked within 50 cM, 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of allele T at marker locus S26383-001-Q001. In certain embodiments, the at least one marker associated with early maturity or early flowering comprises a marker locus within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of the chromosomal interval. In certain embodiments, the at least one marker associated with early maturity or early flowering comprises a marker locus within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of allele T at marker locus S26383-001-Q001.

In certain embodiments, the at least one marker or marker loci associated with early maturity or early flowering or linked to the early maturity or early flowering allele is selected from the group consisting of a T at marker locus S26383-001-Q001, corresponding to a T at position 11441207 on Chr15, a G at position 11430145 on Chr15, a 1 bp deletion at position 11434182 on Chr15, an A at position 11436193 on Chr15, an A at position 11522371 on Chr15, a G at position 11529959 on Chr15, a T at position 11542556 on Chr15, a G at position 11545315 on Chr15 or any combination thereof. In certain embodiments, the at least one marker comprises a T at marker locus S26383-001-Q001. In certain embodiments, at least 2 (e.g., 2, 3, 4, 5, 6, 7, 8, 9, 10, 20 or more) markers are detected. In certain embodiments, the at least 2 markers comprise a haplotype that is associated with early flowering or early maturity.

In certain embodiments, the desired reproductive growth phenotype is late maturity or late flowering. In certain embodiments, the methods for introgressing a late maturity or late flowering phenotype and for producing a subpopulation of soybean plants or soybean germplasm having a late maturity or late flowering comprises crossing a first soybean plant or first soybean germplasm comprising a late maturity or late flowering allele C at marker locus S26383-001-Q001 (position 126 of SEQ ID NO. 2) within a chromosomal interval described herein (e.g., a chromosomal interval flanked by and including BARCSOYSSR_15_0312 and BARCSOYSSR_15_1185), with a second soybean plant or second soybean germplasm to form a soybean plant or soybean germplasm population, genotyping the soybean plant or soybean germplasm population for the presence of at least one marker associated with late maturity or late flowering linked to or within the chromosomal interval, and selecting from the soybean population one or more soybean plants or soybean germplasm comprising the at least one marker associated with late maturity or late flowering. In certain embodiments, the second soybean plant does not comprise the late maturity or late flowering allele C at marker locus S26383-001-Q001. In certain embodiments, the second soybean plant or soybean germplasm comprises an early maturity or early flowering allele T at marker locus S26383-001-Q001 within the chromosomal interval. In certain embodiments, the second soybean plant or soybean germplasm is the same as the first soybean plant or soybean germplasm, such that the crossing step comprises self-fertilization of the first soybean plant or soybean germplasm. In certain embodiments, the one or more soybean plants selected have a later maturity and/or a later flowering as compared to the second soybean plant or soybean germplasm.

In certain embodiments, the at least one marker associated with late maturity or late flowering linked to or within the chromosomal interval is linked within 50 centimorgans (cM), 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of the chromosomal interval. In certain embodiments, the at least one marker associated with late maturity or late flowering linked to or within the chromosomal interval is linked within 50 cM, 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of allele C at marker locus S26383-001-Q001. In certain embodiments, the at least one marker associated with late maturity or late flowering comprises a marker locus within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of the chromosomal interval. In certain embodiments, the at least one marker associated with late maturity or late flowering comprises a marker locus within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of allele C at marker locus S26383-001-Q001.

In certain embodiments, the at least one marker associated with maturity or flowering time linked to or within the chromosomal interval is within 50 centimorgans (cM), 40 cM, 30 cM, 25 cM, 20 cM, 15 cM, 10 cM, 9 cM, 8 cM, 7 cM, 6 cM, 5 cM, 4 cM, 3 cM, 2 cM, or 1 cM of the chromosomal interval or of the allele corresponding to position 126 of SEQ ID NO: 1 (S26383-001-Q001 (Early) marker) or 2 (S26383-001-Q001 (Late) marker). In certain embodiments, the at least one marker associated with maturity or flowering comprises a marker locus within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of the chromosomal interval or of the allele corresponding to position 126 of SEQ ID NO: 1 (S26383-001-Q001 (Early) marker) or 2 (S26383-001-Q001 (Late) marker)

In certain embodiments, the at least one marker or marker loci associated with late maturity or late flowering or linked to late maturity or late flowering allele is selected from the group consisting of a C at marker locus S26383-001-Q001, corresponding to a C at position 11441207 on Chr15, a T at position 11430145 on Chr15, a T at position 11434182 on Chr15, a G at position 11436193 on Chr15, a G at position 11522371 on Chr15, an A at position 11529959 on Chr15, an A at position 11542556 on Chr15, an A at position 11545315 on Chr15 or any combination thereof. In certain embodiments, the at least one marker comprises a C at marker locus S26383-001-Q001. In certain embodiments, at least 2 (e.g., 2, 3, 4, 5, 6, 7, 8, 9, 10, 20 or more) markers are detected. In certain embodiments, the at least 2 markers comprise a haplotype that is associated with late maturity or late flowering.

Also provided herein are method for selecting a population segregating for maturity in plant breeding program, comprising crossing a first soybean plant with a second soybean plant to produce a population, genotyping the population for the presence one or more markers associated with maturity or flowering, and selecting a subpopulation of plants having a marker associated with the desired maturity or flowering phenotype. In certain embodiments, the desired subpopulation has an early maturity or flowering phenotype and the markers used for selecting the plants can be any early maturity or early flowering marker or haplotype described herein (e.g., a T at marker locus 526383-001-Q001). In certain embodiments, the selected late maturity and/or late flowering subpopulation is homozygous for allele T at marker locus 526383-001-Q001. In certain embodiments, the desired subpopulation has a late maturity or flowering phenotype and the markers used for selecting the plants can be any late maturity or early flowering marker or haplotype described herein (e.g., a C at marker locus S26383-001-Q001). In certain embodiments, the selected late maturity and/or late flowering subpopulation is homozygous for allele C at marker locus S26383-001-Q001. In certain embodiments, the first soybean plant and second soybean plant have the same allele at locus S26383-001-Q001. In certain embodiments, the first soybean plant and second soybean plant have a different allele at locus S26383-001-Q001, such that the population produced from the cross is a segregating population for maturity or flowering.

In certain embodiments of the methods described herein, the molecular markers, markers, or marker loci are genotyped using a suitable amplification-based detection method. For example, PCR, RT-PCR, and LCR. PCR, RT-PCR, and LCR are in particularly broad use as amplification and amplification-detection methods for amplifying nucleic acids of interest (e.g., those comprising marker loci) and facilitating detection of the markers. Such nucleic acid amplification techniques can be applied to amplify and/or detect nucleic acids of interest, such as nucleic acids comprising marker loci. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. The primers and probes for use in the methods described herein is not particularly limited and may be designed using methods and/or software known in the art, such as, for example, LASERGENE® or Primer3. It is not intended that the primers be limited to generating an amplicon of any particular size. For example, the primers used to amplify the marker loci and alleles herein are not limited to amplifying the entire region of the relevant locus. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

Non-limiting examples of polynucleotide primers useful in the methods described herein for amplification of at least a portion of one or more genomic regions of the soybean genome (e.g., genomic regions comprising SEQ ID NOs: 1, 2, 43, 44, 45, 46, 47, 48, 49, and 50) comprise SEQ ID NOs: 5, 6, 7, 8, 9, 10, 11, 12, 13, 14, 15, 16, 17, and 18 or any combination thereof. In certain embodiments, SEQ ID NOs: 5 and 6 are used in the methods for detecting the S26383-001-Q001 early and late allele marker.

Non-limiting examples of polynucleotide probes useful for detecting the marker loci comprise SEQ ID NOs: 3 and 4.

In certain embodiments, probes used in detecting the markers described herein will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. Detectable labels may also include reporter-quencher pairs, such as are employed in Molecular Beacon and TaqMan™ probes. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively. Selection of appropriate reporter-quencher pairs for particular probes may be undertaken in accordance with known techniques.

Further, it will be appreciated that amplification is not a requirement for marker detection—for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established.

Real-time amplification assays, including MB or TaqMan™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 11-20 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end.

To effectuate SNP allele detection, a real-time PCR reaction can be performed using primers that amplify the region including the SNP locus, for instance the sequences listed in Table 5, the reaction being performed in the presence of all allele-specific probes for the given SNP locus. By then detecting signal for each detectable label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

Other techniques for detecting SNPs can also be employed, such as allele specific hybridization (ASH). ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

In certain embodiments, the markers described herein are detected by SNP genotyping. Several methods are available for SNP genotyping, including but not limited to, hybridization, primer extension, oligonucleotide ligation, nuclease cleavage, minisequencing, and coded spheres. The KASPar® and Illumina® Detection Systems are additional examples of commercially available marker detection systems. KASPar® is a homogeneous fluorescent genotyping system which utilizes allele specific hybridization and a unique form of allele specific PCR (primer extension) in order to identify genetic markers (e.g., a particular SNP marker lined to or associated with high soybean seed protein content). Illumina® detection systems utilize similar technology in a fixed platform format. The fixed platform utilizes a physical plate that can be created with up to 384 markers. The Illumina® system is created with a single set of markers that cannot be changed and utilizes dyes to indicate marker detection.

These systems and methods represent a wide variety of available detection methods which can be utilized to detect the markers described herein (e.g., marker loci linked to or associated with a desired reproductive growth phenotype, but any other suitable method could also be used).

Also provided are methods for modifying plant maturity or flowering comprising introducing a targeted genetic modification within a chromosomal interval flanked by and including BARCSOYSSR_15_0312 and BARCSOYSSR_15_1185, a chromosomal interval flanked by and including BARCSOYSSR_15_0312 and BARCSOYSSR_15_1184, a chromosomal interval flanked by and including BARCSOYSSR_15_0312 and BARCSOYSSR_15_0732, a chromosomal interval flanked by and including BARCSOYSSR_15_0312 and BARCSOYSSR_15_0731, a chromosomal interval flanked by and including BARCSOYSSR_15_0313 and BARCSOYSSR_15_1185, a chromosomal interval flanked by and including BARCSOYSSR_15_0313 and BARCSOYSSR_15_1184, a chromosomal interval flanked by and including BARCSOYSSR_15_0313 and BARCSOYSSR_15_0732, or a chromosomal interval flanked by and including BARCSOYSSR_15_0313 and BARCSOYSSR_15_0731. In certain embodiments, the targeted genetic modification introduces one or more of the markers or alleles described herein.

Also provided are methods for modifying plant maturity or flowering to produce plants having a desired reproductive growth phenotype. In certain embodiments the desired reproductive growth phenotype is early maturity and/or early flowering time. In certain embodiments, the method for producing plants having an early maturity and/or early flowering time phenotype comprises introducing a targeted genetic modification resulting in the presence of a T at marker locus S26383-001-Q001 (a T corresponding to position 11441207 on Chr15), a G at position 11430145 on Chr15, a 1 bp deletion at position 11434182 on Chr15, an A at position 11436193 on Chr15, an A at position 11522371 on Chr15, a G at position 11529959 on Chr15, a T at position 11542556 on Chr15, or a G at position 11545315 on Chr15, or any combination thereof. In certain embodiments, the method for producing plants having an early maturity and/or early flowering time phenotype comprises introducing a targeted genetic modification resulting in the presence of a T at marker locus S26383-001-Q001, an A at position 11522371 on Chr15, a G at position 11529959 on Chr15, or a T at position 11542556 on Chr15, or any combination thereof. In certain embodiments, the method for producing plants having an early maturity and/or early flowering time phenotype comprises introducing a targeted genetic modification resulting in the presence of a T at marker locus S26383-001-Q001 (a T corresponding to position 11441207 on Chr15). In certain embodiments, the method for producing plants having an early maturity and/or early flowering time phenotype comprises introducing a targeted genetic modification in a gene comprising a sequence that is at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to one or more of SEQ ID NOs: 30-42, the targeted genetic modification increasing expression or activity of the encoded polypeptide. In certain embodiments, the method for producing plants having an early maturity and/or early flowering time phenotype comprises introducing a targeted genetic modification in a gene comprising a sequence that is at least or at least about 50%, 55%, 60%, 65%, 70%7, 75%, 80%, 81%, 82%, 83%, 84%, 85%, 86%, 87%, 88%, 89%, 90%, 91%, 92%, 93%, 94%, 95%, 96%, 97%, 98%, or 99% identical to one or more of SEQ ID NOs: 30-42, the targeted genetic modification decreasing expression or activity of the encoded polypeptide. In certain embodiments, the encoded polypeptide comprises an amino acid sequence that is at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 19-29. In certain embodiments, the method for producing plants having an early maturity and/or early flowering time phenotype comprises introducing into a plant cell a recombinant DNA construct comprising a polynucleotide encoding a polypeptide that is at least or at least about 50%, 55%, 60%, 65%, 70%, 75%, 80%, 81%, 82%, 83% 84%, 85%, 86%, 87%, 88%, 89%, 90%, 91%, 92%, 93%, 94%, 95%, 96%, 97%, 98%, or 99% identical to one or more of SEQ ID NOs: 19-29, optionally operably linked to a heterologous regulatory element and generating a plant. In certain embodiments, the at least one regulatory sequence is a heterologous promoter. The recombinant DNA construct for use in the method may be any recombinant DNA construct provided herein. In certain embodiments the recombinant DNA is expressed by introducing into a plant, plant cell, plant part, seed, and/or grain the recombinant DNA construct, whereby the polypeptide is expressed in the plant, plant cell, plant part, seed, and/or grain. In certain embodiments the recombinant DNA construct is incorporated into the genome of the plant. In certain embodiments, the method for producing plants having an early maturity and/or early flowering time phenotype comprises introducing into a plant cell a silencing construct (e.g., siRNA, miRNA, RNAi) that decreases the expression of a gene comprising a sequence that is at least or at least about 50%, 55%, 60%, 65%, 70%, 75%, 80%, 81%, 82%8, 83%, 84%, 85%, 86%, 87%, 88%, 89%, 90%, 91%, 92%, 93%, 94%, 95%, 96%, 97%, 98%, or 99% identical to one or more of SEQ ID NOs: 30-42.

In certain embodiments the desired reproductive growth phenotype is late maturity and/or late flowering time. In certain embodiments, the method for producing plants having a late maturity and/or late flowering time phenotype comprises introducing a targeted genetic modification resulting in the presence of a C at marker locus S26383-001-Q001 (a C corresponding to position 11441207 on Chr15), a T at position 11430145 on Chr15, a T at position 11434182 on Chr15, a G at position 11436193 on Chr15, a G at position 11522371 on Chr15, an A at position 11529959 on Chr15, an A at position 11542556 on Chr15, an A at position 11545315 on Chr15 or any combination thereof. In certain embodiments, the method for producing plants having a late maturity and/or late flowering time phenotype comprises introducing a targeted genetic modification resulting in the presence of a C at marker locus S26383-001-Q001, a G at position 11522371 on Chr15, an A at position 11529959 on Chr15, or an A at position 11542556 on Chr15, or any combination thereof. In certain embodiments, the method for producing plants having a late maturity and/or late flowering time phenotype comprises introducing a targeted genetic modification resulting in the presence of a C at marker locus S26383-001-Q001. In certain embodiments, the method for producing plants having a late maturity and/or late flowering time comprises introducing a targeted genetic modification in a gene comprising a sequence that is at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to one or more of SEQ ID NOs: 30-42, the targeted genetic modification increasing expression or activity of the encoded polypeptide. In certain embodiments, the method for producing plants having a late maturity and/or late flowering time phenotype comprises introducing a targeted genetic modification in a gene comprising a sequence that is at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to one or more of SEQ ID NOs: 30-42, the targeted genetic modification decreasing expression or activity of the encoded polypeptide. In certain embodiments, the encoded polypeptide comprises an amino acid sequence that is at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to one or more of SEQ ID NOs: 19-29. In certain embodiments, the method for producing plants having a late maturity and/or late flowering time phenotype comprises introducing into a plant cell a recombinant DNA construct comprising a polynucleotide encoding a polypeptide that is at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to one or more of SEQ ID NOs: 19-29, optionally operably linked to a heterologous regulatory element and generating a plant. In certain embodiments, the at least one regulatory sequence is a heterologous promoter. The recombinant DNA construct for use in the method may be any recombinant DNA construct provided herein. In certain embodiments the recombinant DNA is expressed by introducing into a plant, plant cell, plant part, seed, and/or grain the recombinant DNA construct, whereby the polypeptide is expressed in the plant, plant cell, plant part, seed, and/or grain. In certain embodiments the recombinant DNA construct is incorporated into the genome of the plant. In certain embodiments, the method for producing plants having a late maturity and/or late flowering time phenotype comprises introducing into a plant cell a silencing construct (e.g., siRNA, miRNA, RNAi) that decreases the expression of a gene comprising a sequence that is at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to one or more of SEQ ID NOs: 30-42.

As used herein, a “targeted” genetic modification refers to the direct manipulation of an organism's genes. The targeted modification may be introduced using any technique known in the art, such as, for example, genome editing. In certain embodiments, the targeted genetic modification is introduced using gene editing. The type of gene editing for use in the methods described herein is not particularly limited and may be any gene editing method known in the art. In certain embodiments, the gene editing method comprises the use of a polynucleotide-guided endonuclease or a base editing deaminase. In certain embodiments, the gene editing method is selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute. In certain embodiments, the genome edit may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

The process for editing a genomic sequence combining DSB and modification templates generally comprises providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Genome editing using DSB-inducing agents, such as Cas9-gRNA complexes, has been described, for example in U.S. Patent Application US 2015-0082478 A1, WO2015/026886 A1, WO2016007347, and WO201625131 all of which are incorporated by reference herein.

In certain embodiments the genetic modification is introduced without introducing a double strand break using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

In certain embodiments, base editing comprises (i) a catalytically impaired CRISPR-Cas9 mutant that is mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; or (iv) nickase activity to cleave the non-edited DNA strand, followed by cellular DNA repair processes to replace the G-containing DNA strand.

Also provided are soybean plants, plant cells, seeds and grain having an early maturity and/or early flowering phenotype as compared to a control plant, the soybean plants, plant cells, seeds and grain comprising a targeted genetic modification, recombinant DNA construct, or silencing construct described herein. In certain embodiments, the number of days to maturity is decreased by at least 1, 2, 3, 4, 5, 6, 7, 8, 9, or 10 days and less than 15, 14, 13, 12, 11, 10, 9, 8, 7, 6, 5, 4 or 3 days as compared to the control plant. In certain embodiments, the number of days to flowering is decreased by at least 1, 2, 3, 4, 5, 6, 7, 8, 9, or 10 days and less than 15, 14, 13, 12, 11, 10, 9, 8, 7, 6, 5, 4 or 3 days as compared to the control plant. In certain embodiments, the number of days to maturity and flowering is decreased by at least 1, 2, 3, 4, 5, 6, 7, 8, 9, or 10 days and less than 15, 14, 13, 12, 11, 10, 9, 8, 7, 6, 5, 4 or 3 days as compared to the control plant.

Also provided are soybean plants, plant cells, seeds and grain having a late maturity and/or late flowering phenotype as compared to a control plant, the soybean plants, plant cells, seeds and grain comprising a targeted genetic modification, recombinant DNA construct, or silencing construct described herein. In certain embodiments, the number of days to maturity is increased by at least 1, 2, 3, 4, 5, 6, 7, 8, 9, or 10 days and less than 15, 14, 13, 12, 11, 10, 9, 8, 7, 6, 5, 4 or 3 days as compared to the control plant. In certain embodiments, the number of days to flowering is increased by at least 1, 2, 3, 4, 5, 6, 7, 8, 9, or 10 days and less than 15, 14, 13, 12, 11, 10, 9, 8, 7, 6, 5, 4 or 3 days as compared to the control plant. In certain embodiments, the number of days to maturity and flowering is increased by at least 1, 2, 3, 4, 5, 6, 7, 8, 9, or 10 days and less than 15, 14, 13, 12, 11, 10, 9, 8, 7, 6, 5, 4 or 3 days as compared to the control plant.

As used herein “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

“Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

As used herein “operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame.

As used herein “regulatory element” generally refers to a transcriptional regulatory element involved in regulating the transcription of a nucleic acid molecule such as a gene or a target gene. The regulatory element is a nucleic acid and may include a promoter, an enhancer, an intron, expression modulating elements (EMEs), a 5′-untranslated region (5′-UTR, also known as a leader sequence), or a 3′-UTR or a combination thereof. A regulatory element may act in “cis” or “trans”, and generally it acts in “cis”, i.e., it activates expression of genes located on the same nucleic acid molecule, e.g., a chromosome, where the regulatory element is located.

Soybean plants, seeds, tissue cultures, variants and mutants having an early maturity and/or early flowering or late maturity and/or late flowering produced by the methods described herein are also provided. Soybean plants, seeds, tissue cultures, variants and mutants comprising one or more of the marker loci, one or more of the favorable alleles, and/or one or more of the haplotypes and having the desired reproductive phenotype are provided. Also provided are isolated nucleic acids, kits, and systems useful for the identification and/or selection methods disclosed herein.

The following are examples of specific embodiments of some aspects of the invention. The examples are offered for illustrative purposes only and are not intended to limit the scope of the invention in any way.

### Example 1

This example demonstrates QTL mapping to identify flowering and maturity QTLs.

Crosses were made between soybean varieties differing in flowering time and days to maturity. F1 individuals were self-fertilized to the F3:4 or F4:5 generation (Tables 2 and 3). The F3:4 and F4:5 populations were then planted in replicate within environments in North America that corresponded to their expected maturities. The average number of days from planting to R1 and R8 across an F3:4 or F4:5 progeny line was used to evaluate flowering and maturity phenotypes, respectively.

Leaf discs from eight F3:4 or F4:5 plants were pooled per family. Samples comprising genomic DNA were extracted from leaf tissue of each progeny using a rapid HotSHOT DNA extraction method (Truett, G. E., Heeger, P., Mynatt, R. L., Truett, A. A., Walker, J. A. and Warman, M. L. (2000) Preparation of PCR-Quality Mouse Genomic DNA with Hot Sodium Hydroxide and Tris (HotSHOT). BioTechniques 29: 52-53). The DNA was then genotyped using an average of 140 polymorphic TaqMan SNP assays approximately evenly spaced across the soy genome.

Multiple QTL mapping analysis (MQM) was performed using the MQM R/QTL package under recommended parameters as described in, e.g., Broman, K. W. and Sen, S., A guide to QTL mapping with R/qtl. Springer. http://rgtl.org/book (2009); Arends, D. et al., R/qtl: high-throughput multiple QTL mapping, Bioinformatics 26(23):2990-2992 (2010); and Arends, D. et al., Tutorial-Multiple-QTL Mapping (MQM) Analysis for R/qt, http://rgtl.org/tutorials/MQM-tour.pdf (2014). A LOD value of 3.0 or higher was considered evidence of linkage.

QTL mapping within 5 bi-parental populations revealed a region on chromosome 15 between 6,945,336 bp-15,744,128 bp that is associated with variation in days to the initiation of flowering. Three unique parents donated a late flowering allele to these populations while five parents donated an early allele. Similarly, a QTL for days to maturity was identified on chromosome 15 between 6,927,775 bp-40,756,053 bp in 32 different populations. Across these 32 populations, twenty-seven unique parents donated a genomic region conferring a late maturity effect while 20 parents donated an early maturity effect.

The additive effect of the QTL on days to R1 ranged from 0.46-1.2 days and from 0.44-2.27 days on days to R8, suggesting that the QTL has a large impact on reproductive behavior. The variance explained by the flowering QTL ranged from 3.00-6.48% and the maturity QTL ranged from 5.24% to 22.09%. While the average peak of the QTL effect occurs at about 11,093,396 bp and 12,144,704 for flowering and maturity, respectively, the paucity of genotyped markers flanking the QTL and limited population sizes could impact accurate estimation of the true QTL peak position.

Time to flowering and maturity are highly correlated, and genetic factors that control flowering time typically have an impact on maturity. Given the overlap of the QTL regions, it is likely that the observed phenotypes result from allelic variation at the same locus. Thus, for the remainder of the examples, the QTL for flowering and maturity will be considered as the same. Markers to detect the QTL are provided in Table 4.

TABLE 2
QTL mapping for days to flowering using populations grown across
multiple environments reveals a QTL on chromosome 15.

| | | | | | | | Parent | Parent |
|---|---|---|---|---|---|---|---|---|
| | | | | | | | giving | giving |
| | POP | | | | | | late | early |
| Year | Name | LOC | LOD | Var | QTLPeak | Additive | effect | effect |
| 2013 | POP33 | 37 | 5.35 | 7.47 | 11260143 | 0.46 | P48 | P52 |
| 2013 | POP34 | 38 | 3.98 | 10.75 | 11260143 | 0.52 | P48 | P53 |
| 2014 | POP35 | 39 | 6.48 | 6.22 | 14055337 | 1.21 | P49 | P54 |
| 2015 | POP17 | 19 | 5.72 | 5.30 | 13265568 | 1.16 | P16 | P35 |
| 2015 | POP17 | 20 | 8.78 | 15.68 | 10257010 | 2.27 | P16 | P35 |
| 2015 | POP36 | 19 | 3.00 | 6.08 | 11081081 | 0.83 | P16 | P32 |

TABLE 3
QTL mapping for days to R8 using populations grown across
multiple environments reveals a QTL on chromosome 15.

| | | | | | | | Parent | Parent |
|---|---|---|---|---|---|---|---|---|
| | | | | | | | giving | giving |
| | Pop | | QTLPeak | | | | late | early |
| Year | Name | Location | (bp) | LOD | Var | Additive | effect | effect |
| 2014 | POP1 | LOC1 | 11891896 | 5.82 | 5.76 | 0.73 | P1 | P28 |
| 2014 | POP1 | LOC2 | 15560188 | 5.80 | 6.59 | 0.70 | P1 | P28 |
| 2014 | POP2 | LOC3 | 11891896 | 3.71 | 8.08 | 0.78 | P2 | P29 |
| 2014 | POP3 | LOC4 | 13936887 | 4.28 | 6.99 | 0.86 | P3 | P29 |
| 2014 | POP4 | LOC3 | 15750665 | 4.18 | 7.99 | 0.46 | P4 | P30 |
| 2014 | POP5 | LOC5 | 14817939 | 4.79 | 6.42 | 0.56 | P5 | P30 |
| 2014 | POP6 | LOC6 | 15054897 | 8.03 | 9.07 | 0.97 | P6 | P31 |
| 2014 | POP6 | LOC7 | 11795788 | 5.63 | 6.86 | 0.66 | P6 | P31 |
| 2014 | POP7 | LOC8 | 10943311 | 4.67 | 9.14 | 2.27 | P7 | P32 |
| 2014 | POP8 | LOC8 | 12811689 | 5.73 | 5.36 | 1.33 | P8 | P28 |
| 2014 | POP8 | LOC9 | 11891896 | 10.94 | 7.81 | 1.29 | P8 | P28 |
| 2014 | POP8 | LOC1 | 15560188 | 12.63 | 6.80 | 0.95 | P8 | P28 |
| 2014 | POP9 | LOC6 | 12836070 | 10.16 | 16.42 | 1.16 | P6 | P33 |
| 2014 | POP9 | LOC7 | 15691787 | 6.13 | 8.13 | 0.44 | P6 | P33 |
| 2014 | POP10 | LOC3 | 13284429 | 5.15 | 11.31 | 1.23 | P9 | P30 |
| 2014 | POP11 | LOC10 | 12818830 | 11.40 | 5.70 | 0.88 | P10 | P34 |
| 2014 | POP11 | LOC11 | 12818830 | 12.92 | 6.02 | 0.95 | P10 | P34 |
| 2015 | POP12 | LOC12 | 10652153 | 6.36 | 17.12 | 0.77 | P11 | P31 |
| 2015 | POP13 | LOC13 | 11891896 | 5.54 | 7.88 | 2.06 | P12 | P28 |
| 2015 | POP8 | LOC14 | 9620469 | 6.53 | 9.36 | 1.79 | P8 | P28 |
| 2015 | POP8 | LOC15 | 12811689 | 10.77 | 7.61 | 1.08 | P8 | P28 |
| 2015 | POP8 | LOC16 | 11891896 | 8.05 | 5.80 | 0.94 | P8 | P28 |
| 2015 | POP14 | LOC17 | 14102110 | 3.03 | 6.94 | 0.87 | P13 | P29 |
| 2015 | POP15 | LOC17 | 10652153 | 7.57 | 5.24 | 0.91 | P14 | P29 |
| 2015 | POP16 | LOC18 | 13936887 | 6.65 | 6.67 | 0.86 | P15 | P29 |
| 2015 | POP17 | LOC19 | 11010979 | 8.42 | 5.61 | 1.16 | P16 | P35 |
| 2015 | POP18 | LOC19 | 12897426 | 3.25 | 6.45 | 0.60 | P17 | P35 |
| 2015 | POP19 | LOC19 | 11988471 | 9.22 | 10.98 | 1.67 | P18 | P32 |
| 2015 | POP19 | LOC20 | 11320251 | 4.13 | 7.64 | 1.66 | P18 | P32 |
| 2016 | POP20 | LOC21 | 11320251 | 5.99 | 6.43 | 1.43 | P19 | P36 |
| 2016 | POP21 | LOC21 | 13184454 | 5.30 | 11.94 | 1.86 | P20 | P37 |
| 2016 | POP21 | LOC22 | 11633690 | 3.26 | 13.56 | 0.83 | P20 | P37 |
| 2016 | POP21 | LOC23 | 13184454 | 4.19 | 19.69 | 1.85 | P20 | P37 |
| 2016 | POP21 | LOC24 | 13184454 | 3.17 | 6.95 | 0.79 | P20 | P37 |
| 2016 | POP22 | LOC25 | 11735959 | 4.18 | 15.79 | 0.66 | P12 | P38 |
| 2016 | POP23 | LOC26 | 8949187 | 3.27 | 8.52 | 1.27 | P21 | P35 |
| 2016 | POP24 | LOC27 | 14166259 | 3.10 | 5.94 | 1.03 | P22 | P39 |
| 2016 | POP25 | LOC27 | 13108907 | 3.72 | 6.43 | 0.96 | P13 | P40 |
| 2016 | POP26 | LOC28 | 7961485 | 3.42 | 6.03 | 0.46 | P13 | P41 |
| 2016 | POP26 | LOC29 | 11465896 | 5.50 | 8.83 | 1.07 | P13 | P41 |
| 2016 | POP27 | LOC30 | 11465896 | 7.89 | 11.25 | 1.37 | P23 | P42 |
| 2016 | POP27 | LOC29 | 10004504 | 7.12 | 13.02 | 1.89 | P23 | P42 |
| 2016 | POP28 | LOC31 | 10828087 | 7.08 | 15.24 | 1.29 | P24 | P43 |
| 2016 | POP29 | LOC32 | 13792088 | 4.69 | 6.14 | 0.63 | P25 | P44 |
| 2016 | POP29 | LOC33 | 12767711 | 4.18 | 19.63 | 0.92 | P25 | P44 |
| 2017 | POP30 | LOC34 | 10778096 | 5.83 | 22.09 | 1.54 | P26 | P45 |
| 2017 | POP30 | LOC35 | 8819095 | 6.12 | 12.24 | 1.25 | P26 | P45 |
| 2017 | POP31 | LOC34 | 12971380 | 4.06 | 9.44 | 1.30 | P27 | P46 |
| 2016 | POP32 | LOC36 | 9906144 | 5.21 | 12.74 | 1.39 | P13 | P47 |

PopName = the population produced by the breeding pair
LOD = logarithm (base10) of odds
Var = percent variance

TABLE 4
Markers to detect the reproductive growth QTL

| | | Physical | Physical | | |
|---|---|---|---|---|---|
| | Genetic | Position | position | | |
| Marker | Position* | Start | end | Primer 1 | Primer 2 |
| BARCSOYSSR_15_0312 | 33.7 | 6943350 | 6943387 | SEQ ID NO: 7 | SEQ ID NO: 8 |
| BARCSOYSSR_15_0313 | 33.7 | 6980929 | 6980960 | SEQ ID NO: 9 | SEQ ID NO: 10 |
| BARCSOYSSR_15_0731 | 71.4 | 15737341 | 15737364 | SEQ ID NO: 11 | SEQ ID NO: 12 |
| BARCSOYSSR_15_0732 | 71.4 | 15757430 | 15757475 | SEQ ID NO: 13 | SEQ ID NO: 14 |
| BARCSOYSSR_15_1184 | 78.6 | 40697192 | 40697217 | SEQ ID NO: 15 | SEQ ID NO: 16 |
| BARCSOYSSR_15_1185 | 78.6 | 40773413 | 40773438 | SEQ ID NO: 17 | SEQ ID NO: 18 |

*Approximate genetic position based on the public Consensus 4.0 Genetic Map

### Example 2

This example demonstrates using whole-genome shotgun sequencing to uncover SNPs associated with phenotypic effect and to determine parental haplotypes for QTL refinement.

DNA libraries were prepared for whole-genome shotgun sequencing using the standard Illumina TruSeq-V3 chemistry (Illumina.com) on DNA extracted from the parents of the populations in Example 1. Sequencing was performed using the Illumina Hi-Seq 2000. Samples comprising genomic DNA were sequenced to 3-5× coverage (3-5 Gb/DNA library). Paired-end, 101 bp sequencing reads were obtained and used for SNP calling and genotyping. For SNP calling, reads were aligned against the publicly available w82.a2.v1 assembly (https://phytozome-next.jgi.doe.gov/info/Gmax_Wm82_a2_v1) using Bowtie2 (Langmead and Salzberg 2012), and SNPs were called from unique alignments at a coverage of 3 and purity of 0.98.

Ten lines that donated an early effect QTL and one line that donated a late effect were not sequenced due to unavailability of appropriate reference tissue. However, these unavailable lines were derived from 3-4 backcrosses between a donor line to a recurrent parent, and sequencing data from the recurrent parent of each line was used as a proxy for the derived line. One line failed to sequence and was removed from the analysis. Additionally, four lines were removed that showed high heterogeneity within the target region on chromosome 15, which limits proper assignment of alleles to phenotypic effect.

Comparison of SNP calls following re-sequencing revealed that between 11430145-11545315 bp there is a region of genetic contrast that distinguishes the parents that donate a late maturity effect from those that donate an early effect in the studied populations. Outside of this interval, parents can share in-state haplotypes irrespective of their phenotypic association. These data delimit the QTL to the region of genetic contrast between 11430145-11545315 bp. Within this fine-mapped QTL region, 194 SNPs were identified that distinguish the parental soybean varieties associated with either late or early responses to maturity time. Examples of SNPs within the interval that can be used to differentiate and select for a favorable phenotype is demonstrated in Table 5.

TABLE 5
Markers that can differentiate and select for
early or late phenotypic effect on chromosome 15.

| | | | | Early | Late |
|---|---|---|---|---|---|
| | | | | Maturity/ | Maturity/ |
| SEQ ID | Chro- | Position | | Early | Late |
| NO: | mosome | (bp) | Flanking Sequence | Flowering | Flowering |
| 43 | Chr15 | 11430145 | TTTAGAAGAGCCAAATCATA | G | T |
| | | | TAATTCATACATGCTGCAAA | | |
| | | | GATGGATAAT[G/T]GTAGTTG | | |
| | | | TTTACATATGTTTAATATCTA | | |
| | | | AGCTTTCCTTCACTTGGTATT | | |
| | | | G | | |
| 44- | Chr15 | 11434182 | TTTGTTCCTTCCATTATTGAA | InDel | T |
| Early | | | AAGTTTAACCTACGTGACAA | | |
| 45- | | | GTACATGTC[InDel/T]TTTTTT | | |
| Late | | | TTTTTTTTTAATTGTATTATAT | | |
| | | | ATATACAATAACTTAACGTC | | |
| | | | GT | | |
| 46 | Chr15 | 11436193 | GGTAGAATTACATGTGACTG | A | G |
| | | | GGATAGCTTGCCAAATCAGC | | |
| | | | TTGTTCAAAC[A/G]CTTGTCC | | |
| | | | CTTAAGTTGCCTTGTTAGGTG | | |
| | | | AGAGCATAATCTATGACAAC | | |
| | | | AA | | |
| 1- | Chr15 | 11441207 | CTTCTCCAACGACTGTGCGG | T | C |
| Early | | | CCAACTGAAGCTCCATGTTC | | |
| 2- | | | AACTGCAGCCCAAACGCCTG | | |
| Late | | | CATCAGAAACTCACAAGCAT | | |
| | | | ACCTCAAGGGAAAAGGAATA | | |
| | | | CACCTAGCAGAGGTATGGTG | | |
| | | | GCAAA[C/T]AACAAGCCCCCA | | |
| | | | CAACCTCATCGAAGTGCGGC | | |
| | | | CACCAACACCTTCCTCATCAT | | |
| | | | TCCCATTGATAATAACAGCC | | |
| | | | ATCACCAACGACGCAGTCGA | | |
| | | | GCCCATGTTAGCCATATACT | | |
| | | | GAGCATGGCAACCGTGAGGC | | |
| | | | GCCCTGAGCGTGGACCCAAC | | |
| | | | CAAACACAGAGGCTGCACAA | | |
| | | | GAGCCTCATCCTGCACCACC | | |
| | | | CTCACAGCAGAAGCATGACA | | |
| | | | ATCCACAATCA | | |
| 47 | Chr15 | 11522371 | CTTCTTAGTGTGCAAGGGGC | A | G |
| | | | AAGAGCGGTTTCGCGTGAAC | | |
| | | | GATGTCATGC[A/G]GACAAA | | |
| | | | GCCGTAGCTGGGGGGGCGCG | | |
| | | | TGACGTGGCTGGAGGACAGG | | |
| | | | AGTG | | |
| 48 | Chr15 | 11529959 | AGAGAGAACAGGAATTTTTA | G | A |
| | | | GACAATAATCGTGAAGAGCA | | |
| | | | GTTTCCACAT[G/A]TTTATAG | | |
| | | | AGATTGGAGAAGATCTGTCC | | |
| | | | GTAGGGGAAGACGTTTTGAC | | |
| | | | AAG | | |
| 49 | Chr15 | 11542556 | TGTGGAAAGGATAGTTCCAC | T | A |
| | | | GACACAATGGCACCGATAAC | | |
| | | | ACCAAGGGGG[T/A]GAAATT | | |
| | | | CTACTTTGGATCTCTTATGAA | | |
| | | | GCATAGCTCTTCCAGAGGAC | | |
| | | | CTG | | |
| 50 | Chr15 | 11545315 | CTAGCCTACATAAACTCCGC | G | A |
| | | | ACGACATAGATTTGCAATTA | | |
| | | | ATCCGAATTC[G/A]AATTTTA | | |
| | | | AATTCCACATATATAAGCAA | | |
| | | | ATCACACCGATCAAGCAAAA | | |
| | | | CAC | | |

### Example 3

This example demonstrates the development and use of an assay for germplasm characterization and breeding selections.

A SNP at chr15:11,441,207 bp is strongly associated with the flowering and maturity effects across the parents of the hi-parental mapping populations (Tables 6 and 7). A TaqMan assay named S26383-001-Q001 was developed to genotype this SNP and assess the allelic state within germplasm and populations. Samples comprising genomic DNA are isolated from a plurality of plants. Plants with an allele “T” at marker locus S26383-001-Q001 (i.e., position 126 of SEQ ID NO: 1 which corresponds to a T at position 11441207 on Chr15) have an earlier flowering and maturity phenotype from the Chr15 QTL while, while those with an allele “C” at marker locus S26383-001-Q001 (i.e., position 126 of SEQ ID NO: 2 which corresponds to a C at position 11441207 on Chr15) have later flowering and maturity times. S26383-001-Q001 (Early) refers to a T at marker locus S26383-001-Q001, while S26383-001-Q001 (Late) refers to a C at marker locus S26383-001-Q001. A few of the parents were genotyped as heterozygous “C; T”, indicating the early and late alleles may have been segregating in the seed source used for this analysis.

TABLE 6
Association of S26383-001 allelic state with parental
maturity effect in the QTL mapping populations.

| | S26383-001 | |
|---|---|---|
| Parent | Allele | Effect |
| P1 | C; C | Late |
| P2 | C; C | Late |
| P3 | C; C | Late |
| P4 | C; C | Late |
| P5 | C; C | Late |
| P6 | C; C | Late |
| P7 | C; T | Late |
| P8 | C; C | Late |
| P9 | C; C | Late |
| P10 | C; C | Late |
| P11 | C; C | Late |
| P12 | C; T | Late |
| P13 | C; C | Late |
| P14 | C; C | Late |
| P15 | C; C | Late |
| P16 | C; C | Late |
| P17 | C; C | Late |
| P18 | C; C | Late |
| P19 | C; C | Late |
| P20 | C; C | Late |
| P21 | C; T | Late |
| P22 | C; C | Late |
| P23 | C; C | Late |
| P24 | C; C | Late |
| P25 | C; C | Late |
| P26 | C; C | Late |
| P27 | C; C | Late |
| P28 | T; T | Early |
| P29 | T; T | Early |
| P30 | C; T | Early |
| P31 | T; T | Early |
| P32 | T; T | Early |
| P33 | T; T | Early |
| P34 | C; T | Early |
| P35 | T; T | Early |
| P36 | T; T | Early |
| P37 | T; T | Early |
| P38 | T; T | Early |
| P39 | C; T | Early |
| P40 | T; T | Early |
| P41 | T; T | Early |
| P42 | T; T | Early |
| P43 | T; T | Early |
| P44 | C; T | Early |
| P45 | T; T | Early |
| P46 | T; T | Early |
| P47 | C; C | Early |

TABLE 7
Association of S26383-001 allelic state with parental
flowering time effect in the QTL mapping populations.

| | S26383-001 | |
|---|---|---|
| Parent | Allele | Effect |
| P48 | C; C | Late |
| P49 | C; C | Late |
| P16 | C; C | Late |
| P52 | T; T | Early |
| P53 | T; T | Early |
| P54 | T; T | Early |
| P35 | T; T | Early |
| P32 | T; T | Early |

### Example 4

This example demonstrates the use of SNP loci in plant breeding.

The allelic state of S26383-001 can inform parental choices for new breeding crosses. For example, one may desire to limit maturity variation within a population by selecting parents that have the same allele at this locus. Alternatively, one could drive maturity differences in a population by selecting parents that have different alleles at S26383-001. In a segregating population, one could select plants or seed homozygous for either a “T” or “C” allele to derive earlier or later sub-populations of plants, respectively. Additional associated SNPs, genomic variants, or haplotypes, either within or genetically linked to the fine-mapped region could serve as complementary and alternative tools for such processes.

### Example 5

This example describes use of a haplotype to select for early or late reproductive phenotypes.

Genotypes obtained at two or more genomic loci, such as those listed in Table 5, can be used to determine a haplotype that is associated with early or late flowering time or maturity. An example haplotype that could be used to select for a plant with earlier flowering time or maturity could comprise two or more of the following alleles on chromosome 15: a G at position 11430145 bp, a T deletion at position 11434182, an A at position 11436193, a T at position 11441207, an A at position 11522371, a G at position 11529959, a T at position 11542556, and a G at position 11545315. Alternatively, a haplotype comprising two more of the following alleles on chromosome 15 can be used to select a plant with a later maturity: a T at position 11430145 bp, a Tat position 11434182, a Gat position 11436193, a Cat position 11441207, a Gat position 11522371, an A at position 11529959, an A at position 11542556, and an A at position 11545315.

### Example 6

This example demonstrates the identification of putative candidate genes.

There are 11 gene models in the interval between 11430145-11545315 bp following the w82.a2.v1 reference gene set (Table 8). These genes are candidate loci that could confer variation in the maturity and flowering time phenotypes. SNPs were identified within Glyma.15g140000, Glyma.15g140700, Glyma.15g140800, and Glyma.15g140900 that alter the predicted encoded amino acid sequence of each gene and that are perfectly associated with phenotypic effect of the parents of the populations in Example 1 (Table 9).

While each of these genes are potential candidates for controlling flowering and maturity, Glyma.15G140000 is particularly interesting as it encodes a phytochrome B protein, named GmPhyB2 (Wu, F. Q., Zhang, X. M., Li, D. M., & Fu, Y. F. (2011)).

A SNP at position 11441207 bp occurs within the first exon of Glyma.15G140000.1 and creates a non-synonymous mutation that changes a highly conserved valine residue (SEQ ID NO: 20) to an isoleucine residue (V394I) (SEQ ID NO: 21) in the predicted protein sequence (Table 9). Such a mutation could either limit the effect of the protein or create a novel or enhanced function to induce an earlier flowering and maturing plant. A recent study found that when GmPHYB2 is rendered non-functional following a CRISPR/Cas9 induced deletion there is no effect on flowering time (Zhao, Fen, et al. “CRISPR/Cas9-engineered mutation to identify the roles of phytochromes in regulating photomorphogenesis and flowering time in soybean.” The Crop Journal (2022)). If the GmPHYB2 is the gene causing the phenotype observed in Example 1, these results would suggest that the identified mutation either leads to a novel protein function or the phenotypic effect is observable only within a particular genetic or environmental context.

Beyond the SNP variation identified here, there are potentially additional genomic variants (insertions, deletions, copy number variation, etc.) either in the listed genes or within regulatory regions that could lead to the observed phenotypic variation. Similarly, it is possible that there are genes within the interval that are not annotated within the w82.a2.v1 gene set that could cause the phenotype. Validation of the candidate genes can be performed to confirm their function.

TABLE 8
Gene models in the fine-mapped Chr15 QTL interval

| Gene Model | Start | End |
|---|---|---|
| Glyma.15g139900 | 11427754 | 11431219 |
| Glyma.15g140000 | 11435551 | 11442683 |
| Glyma.15g140100 | 11444151 | 11449243 |
| Glyma.15g140200 | 11461119 | 11467932 |
| Glyma.15g140300 | 11476972 | 11483512 |
| Glyma.15g140400 | 11495189 | 11495691 |
| Glyma.15g140500 | 11496139 | 11502209 |
| Glyma.15g140600 | 11510777 | 11511778 |
| Glyma.15g140700 | 11520847 | 11522863 |
| Glyma.15g140800 | 11525771 | 11533394 |
| Glyma.15g140900 | 11538070 | 11545857 |

TABLE 9
Associated SNPs that create missense and splice-site mutations in the predicted
amino acid sequence of genes that occur in the fine-mapped Chr15 QTL interval.

| | Position | Gene model | | | | | |
|---|---|---|---|---|---|---|---|
| Chromosome | (bp) | (w82.a2.v1) | Description | Annotation | W82.a2 | Early | Late |
| Chr15 | 11441207 | Glyma.15G140000.1 | phytochrome | nonsynonymous | C | T | C |
| | | | B (PhyB2) | | | | |
| Chr15 | 11522371 | Glyma.15G140700.1 | HCP-like | splice variant | G | A | G |
| | | | superfamily | | | | |
| | | | protein | | | | |
| Chr15 | 11529959 | Glyma.15G140800.1 | FIP1-Like1 | nonsynonymous | A | G | A |
| | | | protein | | | | |
| Chr15 | 11542556 | Glyma.15G140900.1 | Aldehyde | nonsynonymous | A | T | A |
| | | | dehydrogenase | | | | |

### Example 7

This example demonstrates gene editing for optimal reproductive behavior.

A gene with a role in flowering time or maturity that lies within the QTL interval identified in Example 1 can be manipulated to produce a favorable reproductive phenotype. For example, using gene editing with CRISPR/Cas9, specific mutations could be made to alter the protein coding sequence of a selected gene such that the gene then induces an earlier or later plant maturity. Plant breeders could select upon the maturity variation to produce cultivars that produce higher yields within a target environment. Such novel genetic variation can expand the geographical range of the original cultivar and provide new genetic variation for future breeding crosses. Alternative biotechnological approaches could be envisioned that would use the underlying functional gene to create a favorable phenotype. These may include alteration of regulatory regions to modify gene expression or adding additional or modified copies of the gene.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A method for producing soybean plants or soybean germplasm having an early maturity or early flowering phenotype, the method comprising:
a. crossing a first soybean plant or soybean qermplasm with a second soybean plant or soybean qermplasm to form a soybean plant or soybean germplasm population, wherein the first soybean plant or soybean qermplasm is an elite line;
b. isolating nucleic acids from the soybean plants or soybean germplasm of the population;
c. genotyping the nucleic acids for the presence of at least one marker associated with early maturity or early flowering linked to or within a chromosomal interval flanked by and including marker loci BARCSOYSSR_15_0312 and BARCSOYSSR_15_1185, the chromosomal interval comprising a T at a marker locus corresponding to position 126 of SEQ ID NO: 1; and
d. selecting one or more of the soybean plants or soybean germplasm comprising the at least one marker associated with early maturity or early flowering.

2. The method of claim 1, wherein the at least one marker associated with early maturity or early flowering is linked within 10 centimorgans (cM) of the chromosomal interval.

3. The method of claim 1, wherein the at least one marker is selected from the group consisting of a T at a marker locus corresponding to position 126 of SEQ ID NO: 1, a G at a marker locus corresponding to position 51 of SEQ ID NO: 43, a 1 bp deletion at a marker locus corresponding to position 51 of SEQ ID NO: 45, an A at a marker locus corresponding to position 51 of SEQ ID NO: 46, an A at a marker locus corresponding to position 51 of SEQ ID NO: 47, a G at a marker locus corresponding to position 51 of SEQ ID NO: 48, a T at a marker locus corresponding to position 51 of SEQ ID NO: 49, a G at a marker locus corresponding to position 51 of SEQ ID NO: 50, or any combination thereof.

4. (canceled)

5. The method of claim 1, wherein at least two markers are detected.

6. The method of claim 5, wherein the at least two markers comprise a haplotype that is associated with early maturity or early flowering.

7. The method of claim 1, wherein genotyping comprises amplifying a nucleic acid sequence comprising the at least one marker and detecting the resulting amplified nucleic acid comprising the marker.

8. The method of claim 7, wherein the amplification comprises amplification of at least a portion of one or more genomic regions of the soybean genome comprising SEQ ID NOs: 1, 2, 43, 44, 45, 46, 47, 48, 49, 50 or any combination thereof.

9. The method of claim 8, wherein the amplification comprises providing one or more nucleic acid primers, wherein the nucleic acid primers comprise the nucleic acid sequence selected from the group consisting of SEQ ID NOs: 5, 6, 7, 8, 9, 10, 11, 12, 13, 14, 15, 16, 17, and 18.

10. The method of claim 9, wherein the detecting further comprises hybridization with one or more nucleic acid probes, wherein the nucleic acid probes comprise the nucleic acid sequence selected from the group consisting of SEQ ID NOs: 3 and 4.

11. The method of claim 1, wherein the method further includes producing soybean plants or soybean germplasm having the early maturity or early flowering phenotype by crossing the selected soybean plant or soybean germplasm with a second soybean plant or soybean germplasm to produce a progeny population, wherein at least one soybean plant or soybean germplasm of the progeny population comprises the at least one marker associated with early maturity or early flowering and has an earlier maturity or earlier flowering time as compared to a control plant.

12. A method for producing soybean plants or soybean germplasm having a late maturity or late flowering phenotype, the method comprising:
a. crossing a first soybean plant or soybean qermplasm with a second soybean plant or soybean qermplasm to form a soybean plant or soybean germplasm population, wherein the first soybean plant or soybean qermplasm is an elite line;
b. isolating nucleic acids from the soybean plants or soybean germplasm of the population;
c. genotyping the nucleic acids for the presence of at least one marker associated with late maturity or late flowering linked to or within a chromosomal interval flanked by and including BARCSOYSSR_15_0312 and BARCSOYSSR_15_1185, wherein the chromosomal interval comprises a C at a marker locus corresponding to position 126 of SEQ ID NO: 2; and
d. selecting one or more of the soybean plants or soybean germplasm comprising the at least one marker associated with late maturity or late flowering.

13. The method of claim 12, wherein the at least one marker associated with late maturity or late flowering is linked within 10 centimorgans (cM) of the chromosomal interval.

14. The method of claim 12, wherein the marker is selected from the group consisting of a C at a marker locus corresponding to position 126 of SEQ ID NO: 2, a T at a marker locus corresponding to position 51 of SEQ ID NO: 43, a T at a marker locus corresponding to position 51 of SEQ ID NO: 45, a G at a marker locus corresponding to position 51 of SEQ ID NO: 46, a G at a marker locus corresponding to position 51 of SEQ ID NO: 47, an A at a marker locus corresponding to position 51 of SEQ ID NO: 48, an A at a marker locus corresponding to position 51 of SEQ ID NO: 49, an A at a marker locus corresponding to position 51 of SEQ ID NO: 50, or any combination thereof.

15. (canceled)

16. The method of claim 12, wherein at least two markers are detected.

17. The method of claim 16, wherein the at least two markers comprise a haplotype that is associated with late maturity or late flowering.

18. The method of claim 12, wherein genotyping comprises amplifying a nucleic acid sequence comprising the at least one marker and detecting the resulting amplified nucleic acid comprising the marker.

19. The method of claim 18, wherein the amplification comprises amplification of at least a portion of one or more genomic regions of the soybean genome comprising SEQ ID NOs: 1, 2, 43, 44, 45, 46, 47, 48, 49, 50 or any combination thereof.

20. The method of claim 19, wherein the amplification comprises providing one or more nucleic acid primers, wherein the nucleic acid primers comprise the nucleic acid sequence selected from the group consisting of SEQ ID NOs: 5, 6, 7, 8, 9, 10, 11, 12, 13, 14, 15, 16, 17, and 18.

21. The method of claim 20, wherein the detecting further comprises hybridization with one or more nucleic acid probes, wherein the nucleic acid probes comprise the nucleic acid sequence selected from the group consisting of SEQ ID NOs: 3 and 4.

22. The method of claim 12, wherein the method further includes producing soybean plants or soybean germplasm having the late maturity or late flowering phenotype by crossing the selected soybean plant or soybean germplasm with a second soybean plant or soybean germplasm to produce a progeny population, wherein at least one soybean plant or soybean germplasm of the progeny population comprises the at least one marker associated with late maturity or late flowering and has an later maturity or later flowering time as compared to a control plant.

23-92. (canceled)
